(19) United States
(12) Patent Application Publication
(10) Pub. No.: US 2025/0195092 A1
(43) Pub. Date: Jun. 19, 2025

(54) SYSTEMS AND METHODS FOR REMOVAL OF BLOOD AND THROMBOTIC MATERIAL

(71) Applicant: Walk Vascular, LLC, Irvine, CA (US)

(72) Inventors: Seung Hae Clare Chung, Los Altos, CA (US); Dustin M. Cossio, Brea, CA (US); Gioconda Alvarado, San Jose, CA (US); Connie Vien Cheng, San Jose, CA (US); Nora V. Legarda, San Jose, CA (US); Rafael A. Castellanos Alvarez, San Jose, CA (US)

(21) Appl. No.: 18/976,864

(22) Filed: Dec. 11, 2024

Related U.S. Application Data
(60) Provisional application No. 63/610,567, filed on Dec. 15, 2023.

(51) Int. Cl. A61B 17/22; A61M 39/22; A61M 39/24

(52) U.S. Cl. CPC A61B 17/22; A61M 39/24; A61B 2017/22079; A61M 2039/226; A61M 2039/248

(57) ABSTRACT

A control for use with a system for aspirating thrombus includes an aspiration passage extending between a distal end and a proximal end of the control. The control may comprise a return mechanism comprising a return spring, a solenoid, or a pneumatic connection. Also presented is a control for use with a system for aspirating thrombus comprising a manual valve, including a sliding component, a pinch valve, or a rotary valve.

### CROSS-REFERENCE TO RELATED APPLICATIONS

This application claims priority to and the benefit of U.S. Provisional Patent Application Ser. No. 63/610,567, filed Dec. 15, 2023, and entitled “SYSTEMS AND METHODS FOR REMOVAL OF BLOOD AND THROMBOTIC MATERIAL,” the disclosure of which is incorporated herein by this reference.

### BACKGROUND OF THE INVENTION

### Field of the Invention

The present disclosure pertains generally to medical devices and methods of their use. More particularly, the present invention pertains to aspiration and thrombectomy devices and methods of use thereof.

### Description of the Related Art

Several devices and systems already exist to aid in the removal of thrombotic material. These include simple aspiration tube type devices using vacuum syringes to extract thrombus into the syringe, simple flush-and-aspirate devices, more complex devices with rotating components the pull in, macerate and transport thrombotic material away from the distal tip using a mechanical auger, systems that use very high pressure to macerate the thrombus and create a venturi effect to flush the macerated material away.

All of the devices described above have limitations as a result of individual design characteristics. For example, simple aspiration catheters offer ease of use and rapid deployment but may become blocked or otherwise inoperable when faced with older, more organized thrombotic material. Such devices must be removed and cleared outside the body and then re-inserted into the vasculature, which lengthens the time needed for the procedure and increases the opportunity to kink the catheter shaft. Such kinks may reduce performance by decreasing the cross-sectional area of the catheter or may render the device inoperable.

Mechanical rotary devices use an auger to grab and carry the thrombus away from the target area. Some create transport force via vacuum bottles while others create differential pressure at the distal tip of the device with the auger acting as a low pressure pump. These devices typically work slowly and offer the physician no feedback as to when the device should be advanced further into the lesion.

Flushing type devices include manual flush type devices in which the physician manipulates a hand-driven pump to provide flowing saline at the tip of the device to break up and aspirate the thrombus material, which may introduce performance variations based on the ability of the physician to consistently pump the device over the duration of the procedure. Flushing devices also include high pressure flushing devices that macerate the thrombus and then, using a vortex created by the high pressure fluid, transport the emulsified thrombotic material to a collection bag. These devices are effective at removing all levels of thrombotic material, but the pressure created by the device is so great that its action against certain vessel walls may interrupt the heart muscle stimulation mechanism and create a bradycardia event in certain patients, sometimes requiring that a pacing lead be placed in the patient prior to use. Further, interacting with the thrombotic material outside of the catheter may allow loose material to escape the capture mechanism.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

Implementations of the present invention solve one or more problems in the art with systems, methods, and apparatus for aspirating thrombus. Disclosed are embodiments of a control configured for use in a system for aspirating thrombus, the control having an open position and a closed position, the control comprising a control body having an inlet and an outlet connected by an aspiration passage, a valve disposed within the aspiration passage, an occlusion member disposed at least partially within the control body, wherein the occlusion member is configured to enter the valve and obstruct the aspiration passage, a control interface configured to be manipulated by a user to move the control between the open and closed positions, and a return mechanism configured to facilitate movement of the occlusion member into the valve. The return mechanism may comprise a return spring, a solenoid, or a pneumatic connection. The handle of the control may be positioned at a transverse angle relative to the control body so as to provide a more comfortable grip for holding the control.

In some embodiments, the control may comprise a pinch valve, the control including a control body having a channel disposed therein configured to receive tubing of an aspiration catheter, a first anvil disposed on or near a first side of the channel and a second anvil disposed on or near a second side of the channel opposite the first side of the channel, a control interface configured for manipulation by a user to move the control body between the open position and the closed position, and wherein the distance between the first and second anvils decreases to obstruct the lumen of the tubing when the control is moved from the open position to the closed position.

Also disclosed are embodiments of a control having a valve configured to be manually actuated by a user. In some embodiments, the control may comprise a control body having an inlet and an outlet connected by an aspiration passage configured to direct a fluid through the control, a sliding component configured to control the flow of fluid through the aspiration passage and configured for direct communication with a hand of the user, wherein the control may be placed in a first position by moving the sliding component in a first direction, and wherein the control may be placed in a second position by moving the sliding component in a second direction opposite the first direction.

In some embodiments, the control may comprise a control body having an inlet and an outlet connected by an aspiration passage, a rotary valve disposed at least partially within the aspiration passage and configured to control the flow of fluid through the aspiration passage, and a control interface configured for manipulation by a user to move the rotary valve between an open position and a closed position. In some embodiments, the rotary valve may comprise a quarter-turn ball valve.

In some embodiments, the control may be included in a system for aspirating thrombus, the system comprising an aspiration catheter comprising an elongate shaft configured for placement within a blood vessel of a subject, a supply lumen and an aspiration lumen each extending along the shaft, and an opening at or near a distal end of the supply lumen, the opening configured to allow injection of pressurized fluid into the aspiration lumen at or near the distal end of the aspiration lumen when the pressurized fluid is caused or allowed to flow through the supply lumen, a pressurized fluid source in fluid communication with the supply lumen to provide pressurized fluid to the supply lumen, a vacuum source in fluid communication with the aspiration lumen, and a control configured to regulate application of vacuum to the aspiration catheter, having an open and closed position, the control comprising a control body having an inlet and an outlet connected by an aspiration passage, a valve disposed at least partially within the aspiration passage, an occlusion member disposed at least partially within the control body, wherein the occlusion member is configured to enter the valve and obstruct flow of a fluid through the aspiration passage, a control interface configured to be manipulated by a user to move the control between the open and closed positions, and a return mechanism configured to facilitate movement of the occlusion member into the valve.

In some embodiments the system for aspirating thrombus may comprise an aspiration catheter comprising an elongate shaft configured for placement within a blood vessel of a subject, a supply lumen and an aspiration lumen each extending along the shaft, and an opening at or near a distal end of the supply lumen, the opening configured to allow injection of pressurized fluid into the aspiration lumen at or near the distal end of the aspiration lumen when the pressurized fluid is caused or allowed to flow through the supply lumen, a pressurized fluid source in fluid communication with the supply lumen to provide pressurized fluid to the supply lumen, a vacuum source in fluid communication with the aspiration lumen, and a control configured to regulate application of vacuum to the aspiration catheter, having an open and closed position, the control comprising a control body having a channel disposed therein configured to receive tubing of an aspiration catheter, a first anvil disposed on or near a first side of the channel and a second anvil disposed on or near a second side of the channel opposite the first side of the channel, a control interface configured for manipulation by a user to move the control body between the open position and the closed position, and wherein a distance between the first and second anvils decreases to obstruct flow of a fluid through the lumen of tubing disposed in the channel when the control is moved to the closed position.

In some embodiments, the system for aspirating thrombus may comprise an aspiration catheter comprising an elongate shaft configured for placement within a blood vessel of a subject, a supply lumen and an aspiration lumen each extending along the shaft, and an opening at or near a distal end of the supply lumen, the opening configured to allow injection of pressurized fluid into the aspiration lumen at or near the distal end of the aspiration lumen when the pressurized fluid is caused or allowed to flow through the supply lumen, a pressurized fluid source in fluid communication with the supply lumen to provide pressurized fluid to the supply lumen, and a vacuum source in fluid communication with the aspiration lumen, a control configured to regulate application of vacuum to the aspiration catheter, having an open and closed position, the control comprising a control body having an inlet and an outlet connected by an aspiration passage, a valve disposed at least partially within the aspiration passage configured to be manually actuated by a user, an occlusion member disposed at least partially within the control body, wherein the occlusion member is configured to enter the valve and obstruct flow of a fluid through the aspiration passage, a control interface configured to be manipulated by a user to move the control between the open and closed positions.

Additional features and advantages of exemplary implementations of the invention will be set forth in the description which follows, and in part will be obvious from the description, or may be learned by the practice of such exemplary implementations. The features and advantages of such implementations may be realized and obtained by means of the instruments and combinations particularly pointed out in the appended claims. These and other features will become more fully apparent from the following description and appended claims or may be learned by the practice of such exemplary implementations as set forth hereinafter.

### BRIEF DESCRIPTION OF THE DRAWINGS

FIG. 1 is a plan view of a system for aspirating thrombus according to an embodiment of the present disclosure.

FIG. 2 is a sectional view of a distal end of the aspiration catheter of the system for aspirating thrombus of FIG. 1.

FIG. 3 is a detail view of a y-connector of the aspiration catheter of the system for aspirating thrombus of FIG. 1.

FIG. 4 is a plan view of a system for aspirating thrombus according to an embodiment of the present disclosure.

FIG. 5 is a perspective view of the system for aspirating thrombus of FIG. 4.

FIG. 6 is a perspective view of another aspiration catheter according to an embodiment of the present disclosure.

FIG. 7 is a cross-sectional view of another aspiration catheter according to the presentation disclosure.

FIG. 8 is a sectional view of an exemplary distal end of the aspiration catheter of FIGS. 6 and 7 aspirating thrombus according to the present disclosure.

FIG. 9 is a perspective view of an aspiration tubing set with a control, according to an embodiment of the present disclosure.

FIG. 10 is a plan view of the aspiration tubing sent and control of FIG. 6.

FIG. 11 is an exploded view of the control of FIGS. 9 and 10.

FIG. 12 is an exploded view of some internal components of the control of FIGS. 9-11.

FIG. 13A is a sectional view of the control in a closed position, according to an embodiment of the present disclosure.

FIG. 13B is a sectional view of the control in an open position, according to an embodiment of the present disclosure.

FIG. 14A is a sectional view of a control in a closed position, according to an alternative embodiment of the present disclosure.

FIG. 14B is a sectional view of a control in an open position, according to an alternative embodiment of the present disclosure.

FIG. 15 is a sectional view of a control in a closed position, according to an alternative embodiment of the present disclosure.

FIG. 16 a sectional view of a control in a closed position, according to an alternative embodiment of the present disclosure.

FIG. 17 is a plan view of a control, according to an alternative embodiment of the present disclosure.

FIG. 18 is a perspective view of a control, according to an alternative embodiment of the present disclosure.

FIGS. 19A-19C are plan views of a control having a pinch valve, according to an alternative embodiment of the present disclosure.

FIG. 20 is a plan view of a control having a pinch valve, according to an alternative embodiment of the present disclosure.

FIG. 21A is a plan view of a control, according to an alternative embodiment of the present disclosure.

FIG. 21B is a front view of the control of FIG. 21A.

FIG. 22A is a perspective view of a control, according to an alternative embodiment of the present disclosure.

FIGS. 22B-22C are sectional views of the control of FIG. 22A.

FIG. 23 is a sectional view of a control, according to an alternative embodiment of the present disclosure.

### DETAILED DESCRIPTION

One or more specific embodiments of the present disclosure will be described below. In an effort to provide a concise description of these embodiments, some features of an actual embodiment may be described in the specification. It should be appreciated that in the development of any such actual embodiment, as in any engineering or design project, numerous embodiment-specific decisions will be made to achieve the developers' specific goals, such as compliance with system-related and business-related constraints, which may vary from one embodiment to another. It should further be appreciated that such a development effort might be complex and time consuming, but would nevertheless be a routine undertaking of design, fabrication, and manufacture for those of ordinary skill having the benefit of this disclosure.

One or more embodiments of the present disclosure may generally relate to systems, methods, and apparatus for aspirating thrombus, such as through using a control to regulate the flow of aspirant through an aspiration lumen of the aspiration catheter. The aspiration lumen through the control may comprise a valve having an occlusion member that enters the valve to obstruct the aspiration lumen and place the control in a closed position. The occlusion member may comprise a piston structure having a plunger, a sliding component, one or more anvils of a pinch valve, or a rotary valve. The control may also comprise a return mechanism configured to maintain the control in the closed position. The return mechanism may include a return spring, solenoid, or pneumatic connection. While the present disclosure will describe a particular implementation of various occlusion members and return mechanisms, it should be understood that the devices, systems, and methods described herein may be applicable to other uses. Additionally, elements described in relation to any embodiment depicted and/or described herein may be combinable with elements described in relation to any other embodiment depicted and/or described herein.

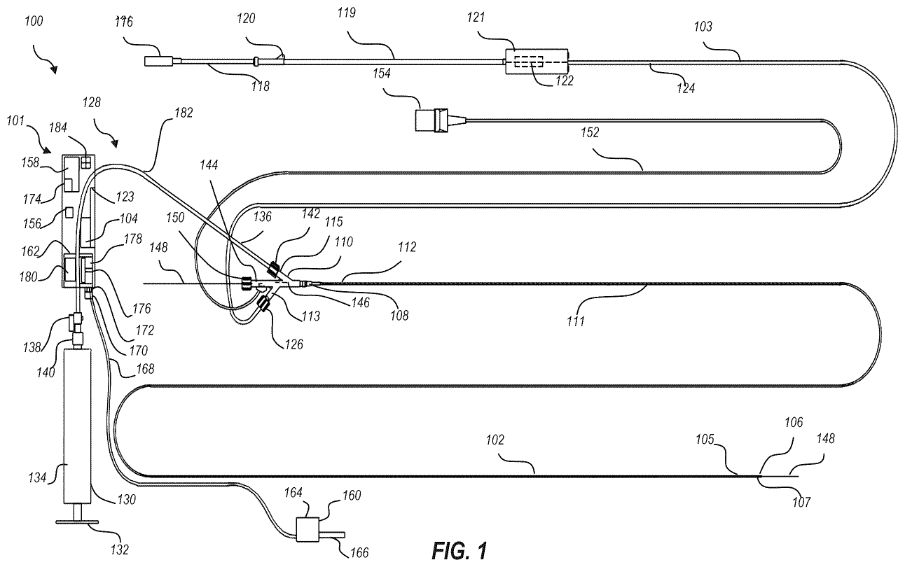

A system for aspirating thrombus 100 is illustrated in FIG. 1. The system for aspirating thrombus 100 includes three major components: a pump 101, an aspiration catheter 102, and a tubing set 103. The aspiration catheter 102 and the tubing set 103 represent disposable components 104, and the pump 101, and the pump's associated pump base, is a reusable component. It is not necessary to sterilize the pump 101 as it may be kept in a non-sterile field or area during use. The aspiration catheter 102 and the tubing set 103 may each be supplied sterile, after sterilization by ethylene oxide gas, electron beam, gamma, or other sterilization methods. The aspiration catheter 102 may be packaged and supplied separately from the tubing set 103, or the aspiration catheter 102 and the tubing set 103 may be packaged together and supplied together. Alternatively, the aspiration catheter 102 and tubing set 103 may be packaged separately, but supplied together (i.e., bundled). The aspiration catheter 102 has a distal end 105 and includes an over-the-wire guidewire lumen/aspiration lumen 106 extending between an open distal end 107, and a proximal end 108 comprising a y-connector 110. While reference is made to the aspiration lumen 106 being an “over-the-wire guidewire lumen” it will be understood that the distal end 105 can include a lumen for rapid-exchange such that a guidewire need not be disposed within the aspiration lumen 106.

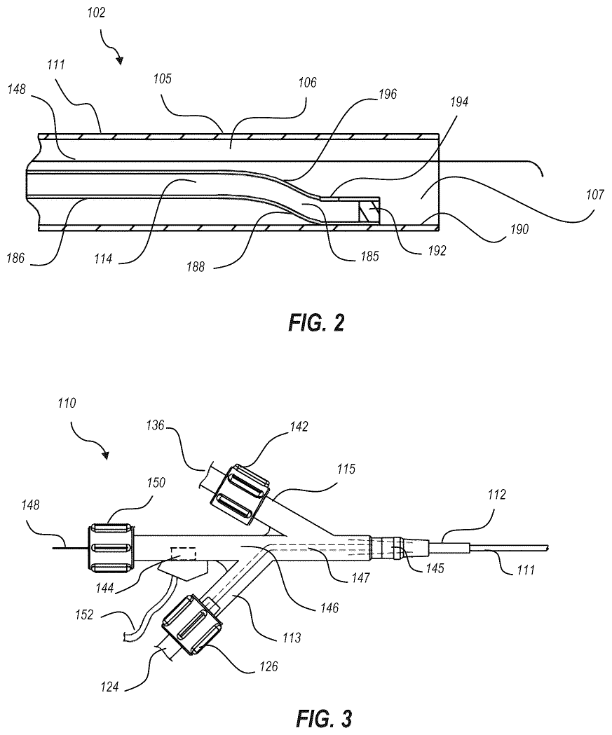

The catheter shaft 111 of the aspiration catheter 102 is connected to the y-connector 110 via a protective strain relief 112. In other embodiments, the catheter shaft 111 may be attached to the y-connector 110 with a luer fitting. The y-connector 110 comprises a first female luer 113 which communicates with a catheter supply lumen 114 (FIG. 2), and a second female luer 115 which communicates with the guidewire lumen/aspiration lumen 106.

A spike 116 for coupling to a fluid source 20 (e.g., saline bag, saline bottle) allows fluid to enter through an extension tubing 118 and flow into a supply tube 119. An optional injection port 120 allows injection of materials or removal of air. A cassette 121 having a moveable piston 122 is used in conjunction with a mechanical actuator 123 of the pump 101. Fluid is pumped into an injection tube 124 from action of the cassette 121 as applied by the actuator 123 of the pump 101. A male luer 126, hydraulically communicating with the catheter supply lumen 114, via the injection tube 124, is configured to attach to the female luer 113 of the y-connector 110.

Accessories 128 are illustrated that are intended for applying a vacuum source, such as a syringe 130 having a plunger 132 and a barrel 134, to the aspiration lumen 106 of the catheter 102. The syringe 130 is attached to a vacuum line 136 via the luer 140 of the syringe 130. A stopcock 138 may be used on the luer 140 to maintain the vacuum, or alternatively, the plunger 132 may be a locking variety of plunger that is configured to be locked in the retracted (vacuum) position. A male luer 142 at the end of the vacuum line 136 may be detachably secured to the female luer 115 of the y-connector 110 of the aspiration catheter 102. As shown in more detail in FIG. 3, a pressure sensor or transducer 144 is secured inside an internal cavity 146 of the y-connector 110 proximal to the female luer 113 and the female luer 115. A valve 150, for example a Touhy-Borst, at the proximal end of the y-connector 110 allows hemostasis of the guidewire lumen/aspiration lumen 106 around a guidewire 148. In other embodiments, the valve 150 may comprise a longitudinally spring-loaded seal. The guidewire 148 may be inserted entirely through the guidewire lumen/aspiration lumen 106. Signals output from the pressure sensor 144 are carried through a cable 152 to a connector 154. The connector 154 is plugged into a socket 156 of the pump 101. Pressure related signals may be processed by a circuit board 158 of the pump 101. The pressure transducer 144 may be powered from the pump 101, via the cable 152. The accessories 128 may also be supplied sterile to the user.

A foot pedal 160 is configured to operate a pinch valve 162 for occluding or opening the vacuum line 136. The foot pedal 160 comprises a base 164 and a pedal 166 and is configured to be placed in a non-sterile area, such as on the floor, under the procedure table/bed. The user steps on the pedal 166 causing a signal to be sent along a cable 168 which is connected via a plug 170 to an input jack 172 in the pump 101. The vacuum line 136 extends through a portion of the pump 101. The circuit board 158 of the pump may include a controller 174 configured to receive one or more signals indicating on or off from the foot pedal 160. The controller 174 of the circuit board 158 may be configured to cause an actuator 176 carried by the pump 101 to move longitudinally to compress and occlude the vacuum line 136 between an actuator head 178 attached to the actuator 176 and an anvil 180, also carried by the pump 101. By stepping on the pedal 166, the user is able to thus occlude the vacuum line 136, stopping the application of a negative pressure. In some embodiments, as the pedal 166 of the foot pedal 160 is depressed, the controller may be configured to open the pinch valve 162.

The pressure transducer 144 thus senses a negative pressure and sends a signal, causing the controller to start the motor 182 of the pump 101. As the effect via the electronics is substantially immediate, the motor 182 starts pumping almost immediately after the pedal 166 is depressed. As the pedal 166 of the foot pedal 160 is released, the controller 174 then causes the pinch valve 162 to close. The pressure transducer 144 thus senses that no negative pressure is present and the controller 174 causes the motor 182 of the pump 101 to shut off. Again, the effect via the electronics is substantially immediate, and thus the motor 182 stops pumping almost immediately after the pedal 166 is depressed. During sterile procedures, the main interventionalist is usually “scrubbed” such that the hands only touch items in the sterile field. However, the feet/shoes/shoe covers are not in the sterile field. Thus again, a single user may operate a switch (via the pedal 166) while also manipulating the catheter 102 and guidewire 148. However, this time, it is the sterile field hands and non-sterile field feet that are used. Alternatively, the foot pedal 160 may comprise two pedals, one for occlude and one for open. In an alternative foot pedal embodiment, the pedal 166 may operate a pneumatic line to cause a pressure activated valve or a cuff to occlude and open the vacuum line 136, for example, by forcing the actuator head 178 to move. In another alternative embodiment, the pedal 166 may turn, slide, or otherwise move a mechanical element, such as a flexible pull cable or push rod that is coupled to the actuator 176, to move the actuator head 178. The cable 168 may be supplied sterile and connected to the base 164 prior to a procedure. The occlusion and opening of the vacuum line 136 thus acts as an on and off switch for the pump 101 (via the pressure sensor 144). The on/off function may thus be performed by a user whose hands can focus on manipulating sterile catheters, guidewires, and accessories, and whose foot can turn the pump on and off in a non-sterile environment. This allows a single user to control the entire operation or the majority of operation of the system for aspirating thrombus 100. This can be an advantage both in terms of a rapid, synchronized procedure, but is also helpful in laboratories where additional assistants are not available. The actuator 176 and anvil 180 may be controlled to compress the vacuum line 136 with a particular force, and the actuator 176 may be controlled to move at a particular speed, either when compressing or when removing compression. Speed and force control allows appropriate response time, but may also be able to add durability to the vacuum line 136, for example, by not over compressing. The foot pedal 160 may communicate with the pinch valve 162 via a wired connection through the pump 101 or may communicate with the pinch valve 162 wirelessly. Additionally, or alternatively, the pump may be controlled by buttons 184.

It should be noted that in certain embodiments, the pinch valve 162 and the foot pedal 160 may be incorporated for on/off operation of the pinch valve 162 on the vacuum line 136, without utilizing the pressure sensor 144. In fact, in some embodiments, the pressure sensor 144 may even be absent from the system for aspirating thrombus 100, the foot pedal 160 being used as a predominant control means.

Turning to FIG. 2, a supply tube 186, which contains the catheter supply lumen 114, freely and coaxially extends within the over-the-wire guidewire lumen/aspiration lumen 106. At least a distal end 188 of the supply tube 186 is secured to an interior wall 190 of the guidewire lumen/aspiration lumen 106 of the catheter shaft 111 by adhesive, epoxy, hot melt, thermal bonding, or other securement modalities. A plug 192 is secured within the catheter supply lumen 114 at the distal end 188 of the supply tube 186. The plug 192 blocks the exit of pressurized fluid, and thus the pressurized fluid is forced to exit through an orifice 194 in the wall 196 of the supply tube 186. The free, coaxial relationship between the supply tube 186 and the catheter shaft 111 along their respective lengths, allows for improved flexibility. In some embodiments, in which a stiffer proximal end of the aspiration catheter 102 is desired (e.g., for pushability or even torquability), the supply tube 186 may be secured to the interior wall 190 of the guidewire lumen/aspiration lumen 106 of the catheter shaft 111 along a proximal portion of the aspiration catheter 102, but not along a distal portion. This may be appropriate if, for example, the proximal portion of the aspiration catheter 102 is not required to track through tortuous vasculature, but the distal portion is required to track through tortuous vasculature. The free, substantially unconnected, coaxial relationship between the supply tube 186 and the catheter shaft 111 along their respective lengths, may also be utilized to optimize flow through the guidewire lumen/aspiration lumen 106, as the supply tube 186 is capable of moving out of the way due to the forces of flow (e.g., of thrombus/saline) over its external surface, such that the remaining inner luminal space of the guidewire lumen/aspiration lumen 106 self-optimizes, moving toward the lowest energy condition (least fluid resistance) or toward the largest cross-sectional space condition (e.g., for accommodating and passing pieces of thrombus).

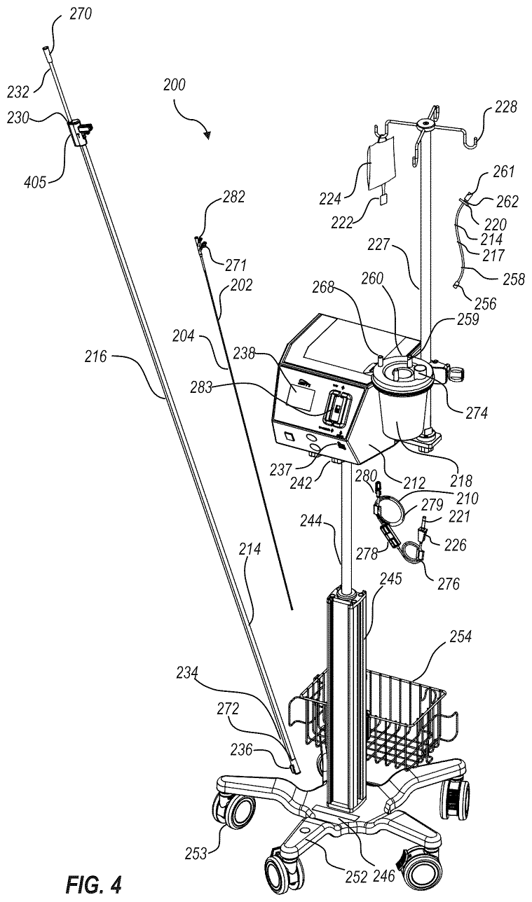

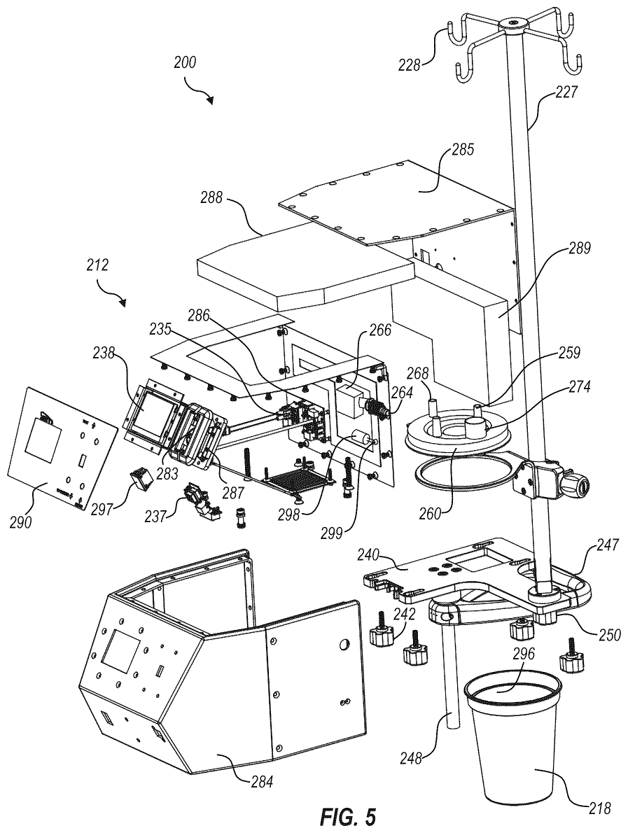

A system for aspirating thrombus 200 is illustrated in FIGS. 4-5. An aspiration catheter 202 is similar to the aspiration catheter 102 of FIGS. 1-3. The aspiration catheter 202 is configured for aspirating thrombus from peripheral vessels, but may also be configured with a size for treating coronary, cerebral, pulmonary or other arteries, or veins. The aspiration catheter 202/system 200 may be used in interventional procedures, but may also be used in surgical procedures. The aspiration catheter 202/system 200 may be used in vascular procedures, or non-vascular procedures (other body lumens, ducts, or cavities). The catheter 202 comprises an elongate shaft 204 configured for placement within a blood vessel of a subject; a catheter supply lumen 114 (FIG. 2) and a guidewire/aspiration lumen 106, each extending along the shaft, the supply lumen 114 having a proximal end 147 and a distal end 185, and the aspiration lumen 106 having a proximal end 145 (FIG. 3) and an open distal end 107 (FIG. 2); and an orifice 194 at or near the distal end 185 of the supply lumen 114, the opening configured to allow the injection of pressurized fluid into the aspiration lumen 106 at or near the distal end 107 of the aspiration lumen 106 when the pressurized fluid is pumped through the supply lumen 114. In some embodiments, the orifice 194 may be located proximal to the distal end 185 of the supply lumen 114. In some embodiments, the distal end 185 of the supply lumen 114 may comprise a plug 192. A pump set 210 (e.g., tubing set) is configured to hydraulically couple the supply lumen 114 to a pump within a saline drive unit (SDU) 212, for injecting pressurized fluid (e.g., saline, heparinized saline) through the supply lumen 114. Suction tubing 214, comprising sterile suction tubing 216 and non-sterile suction tubing 217, is configured to hydraulically couple a vacuum canister 218 to the aspiration lumen 106. A filter 220 may be carried in-line on the suction tubing 214, for example, connected between the sterile suction tubing 216 and the non-sterile suction tubing 217, or on the non-sterile suction tubing 217. The filter 220 is configured to capture large elements such as large pieces of thrombus or emboli.

The pump set 210 includes a saline spike 221 for connection to a port 222 of a saline bag 224, and an inline drip chamber 226 for visually assessing the movement of saline, as well as keeping air out of the fluid being injected. The saline bag 224 may be hung on an IV pole 227 on one or more hooks 228. A pressure sensor 230 such as a vacuum sensor may be used within any lumen of the pump set 210, the suction tubing 214, the supply lumen 114 or aspiration lumen 106 of the catheter 202, or any other component which may see fluid flow. The pressure sensor 230 is shown in FIG. 4 within a lumen at a junction between a first aspiration tube 232 and a control 405. A cable 234 carries signals output from the pressure sensor 230 to a controller 235 in the SDU 212. A connector 236, electrically connected to the cable 234, is configured to be detachably coupled to a mating receptacle 237 (e.g., input jack) in the SDU 212. The SDU 212 also may have a display 238, including an LCD screen or alternative screen or monitor, in order to visually monitor parameters and status of a procedure. In alternative embodiments, the pressure sensor 230 may be replaced by another type of sensor that is configured to characterize fluid flow. In some embodiments, the sensor is a flow sensor, such as a Doppler flow velocity sensor.

The SDU 212 is held on a mount 240 by four locking knobs 242. The mount 240 is secured to a telescoping rod 244 that is adjustable from a cart base 245 via a cart height adjustment knob or other element 246. The mount 240 and a handle 247 are secured to the rod 244 via an inner post 248 that is insertable and securable within an inner cavity in the rod 244. The IV pole 227 secures to the mount 240 via a connector 250. The base 245 may include legs 252 having wheels 253 (e.g., three or more wheels or four or more wheels) and may be movable via the handle 247. The system 200 may also carry a basket 254 for placement of components, products, documentation, or other items.

In use, a user connects a first connector 256 at a first end 258 of the non-sterile suction tubing 217 to a second port 259 on the lid 260 of the canister 218, and connects a second connector 261 at a second end 262 of the non-sterile suction tubing 217 to a vacuum pump input 264 in the SDU 212. A vacuum pump 266 may be carried within the SDU 212 in order to maintain a vacuum/negative pressure within the canister 218. Alternatively, the vacuum inside the canister 218 may be maintained manually, without a vacuum pump, by evacuating the canister 218 via one or more additional ports 268. A user connects a first connector 270 of the sterile suction tubing 216 to an aspiration luer 271 of the aspiration catheter 202 (similar to luer 115), and connects the second connector 272 of the sterile suction tubing 216 to port 274 in the lid 260 of the canister 218. Connector 236 is then coupled to the mating receptacle 237 in the SDU 212 for communication with the control 405 and/or the pressure sensor 230. For instance, the connector 236 can be snapped into mating receptacle 237 in the SDU 212 for communication with elements of the control 405 and/or for communication with the pressure sensor 230, either via cable 234, and/or additional cables or wires. Alternatively, the connector 236 may couple to the mating receptacle 237 by clipping, friction fitting, vacuum fitting, or other means.

After allowing saline to purge through the supply tube 276, cassette 278, and injection tube 279 of the pump set 210, the user connects the luer connector 280 of the pump set 210 to a luer 282 of the aspiration catheter 202 (similar to luer 113). The cassette 278 (similar to cassette 121) is then attached to a saddle 283 in the SDU 212. The saddle 283 is configured to reciprocate a piston to inject the saline from the IV bag 224 at high pressure, after the cassette 121 is snapped in place, keeping the internal contents (e.g., saline) sterile. Systems configured for performing this type of sterile injection of high-pressure saline are described in U.S. Pat. No. 9,883,877, issued Feb. 6, 102, and entitled, “Systems and Methods for Removal of Blood and Thrombotic Material”, which is incorporated by reference in its entirety for all purposes. The SDU 212 is enclosed within a case 284 and a case lid 285. The controller 235 may reside on a circuit board 286. Noise from a motor 287 controlling the saddle 283 and from the vacuum pump 266 is abated by internal foam sections 288, 289. The saddle 283 may be moved directly by the motor 287, or may be moved with pneumatics, using a cycled pressurization. An interface panel 290 provides one or more switches 297 and the display 238. Alternatively, the cassette 121 may couple to the saddle 283 by clipping, friction fitting, vacuum fitting, or other means.

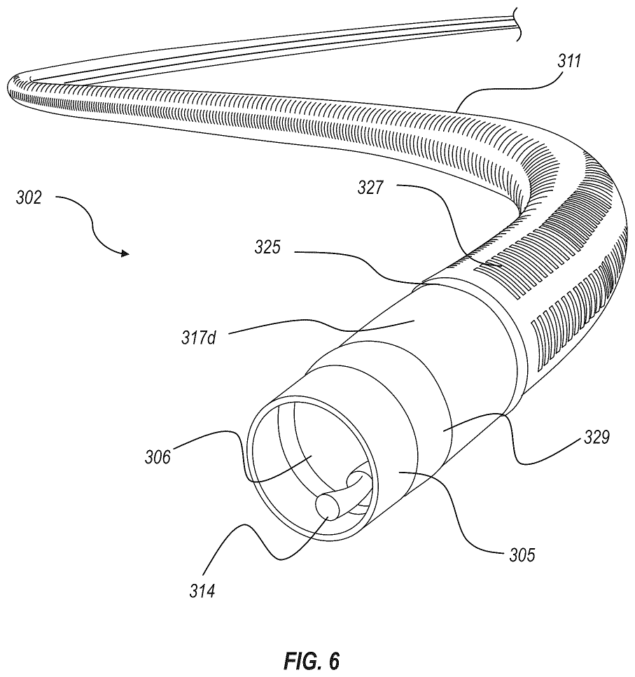

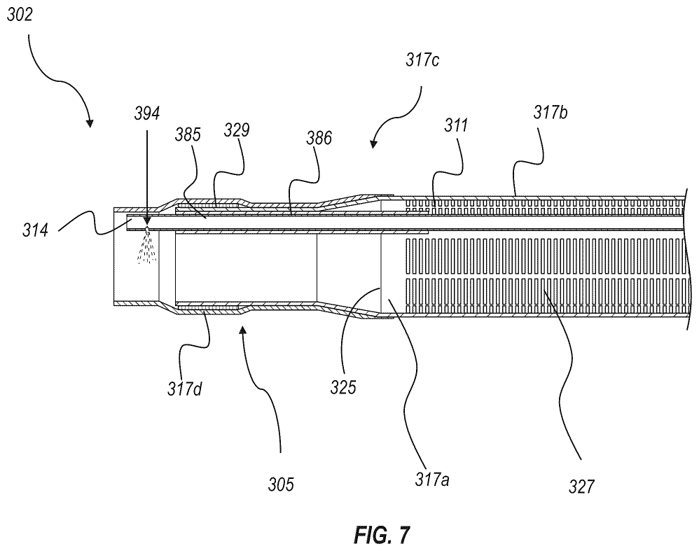

Turning to FIGS. 6-8 illustrated is another configuration of an aspiration catheter 302 that can be used with the systems 100 or 200 for aspirating thrombus. As such, the discussions related to the aspiration catheter 102 and the aspiration catheter 202 are also applicable to the aspiration catheter 302 illustrated in FIGS. 6-8.

As shown in more detail in FIGS. 6-8, aspiration catheter 302 includes an aspiration lumen 306 formed by a shaft 311, such as a hypotube, jacketed by a polymer jacket. For instance, a shaft body 317a is illustrated being jacket by a jacket 317b. A distal end 305 of the aspiration catheter 302 includes a multilayer structure. A portion 317c of the jacket 317b extends distally of a shaft distal end 325 of the shaft 311 to form part of the distal end 305. An outer jacket or layer 317d overlaps the jacket 317b, extends towards and overlaps a shaft distal end 325, and forms the aspiration catheter distal end 305 or the distal most end of the aspiration catheter. The outer jacket 317d protects a distal portion 385 of a supply tube 386 containing a supply lumen 314. While reference is made of a multilayer structure, it will be understood that one or more layers can be omitted or combined together. Additionally, one or more of the layers or shaft body can include braided or other members to increase strength and/or flexibility. Alternatively, the shaft and associated layers can be formed by extruding the shaft or using other structure to form the shaft.

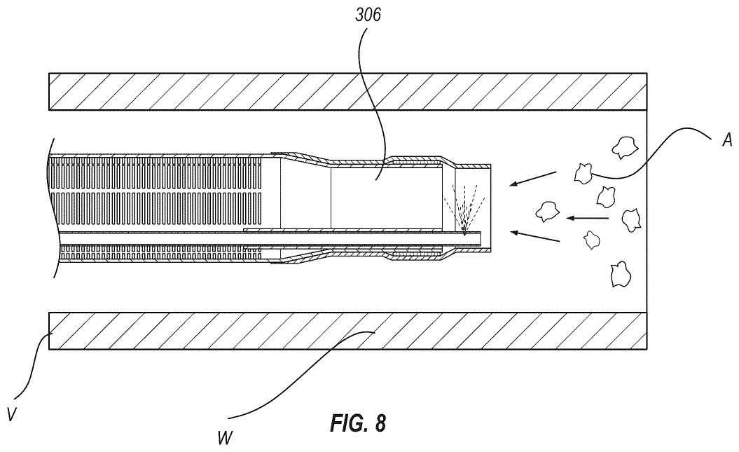

The supply lumen 314 may be configured to provide a high pressure fluid injection, such as saline, within the aspiration lumen 306 for macerating a thrombus as it is aspirated, such as illustrated in FIG. 8 where the aspiration catheter 302 is disposed within a vessel lumen W of a vessel V and aspirant A is being drawing into the aspiration lumen 306. The saline injection may occur through orifice 394 near the distal end of the supply lumen 314 if the opening of the supply lumen is plugged with a plug similar to plug 192 (FIG. 2). Aspiration catheter 302 may also include a radiopaque (RO) ring 329 at or near the distal end 305 of aspiration catheter 302 for identifying the location of aspiration. In the illustrated configuration, the RO ring 329 is disposed between the jacket 317b and outer jacket 317d. The RO ring 329 can be formed of any suitable radiopaque material, such as tantalum, tungsten, platinum/iridium, gold, silver, and combinations or modifications thereof.

The shaft 311 can include one or more openings 327 to increase a flexibility of shaft 311 to aid with advancement of the aspiration catheter 302 through the tortuous anatomy of a patient. While reference is made to a “hypotube,” it will be understood that other tubular structures can be used for the shaft 311. Additionally, the shaft 311 can be formed from polymers, metals, alloys, braided structures, coiled structures, and combinations or modifications thereof. Furthermore, the jacket 317b and outer jacket 317d can be formed of a variety of polymers and copolymers, plastics, PEBAX, HYTREL, rubber, thermoplastic elastomer, other elastomer and combinations or modifications thereof.

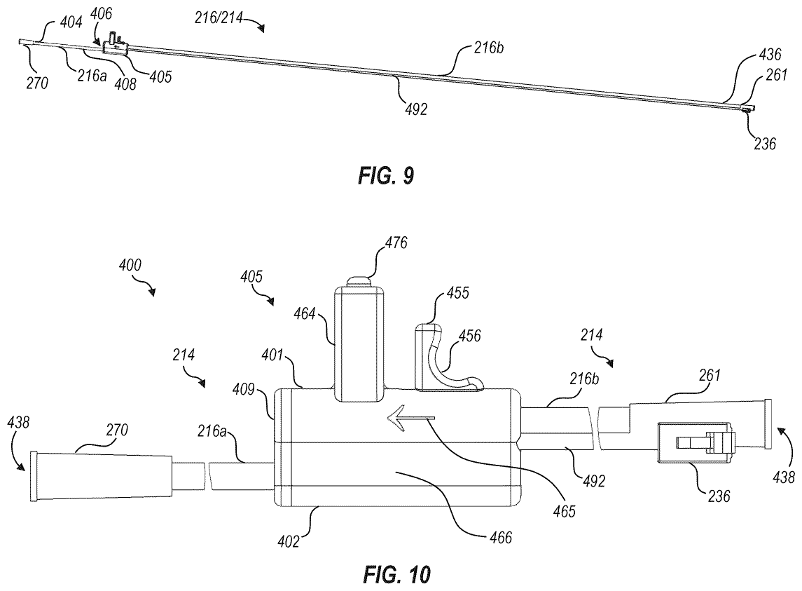

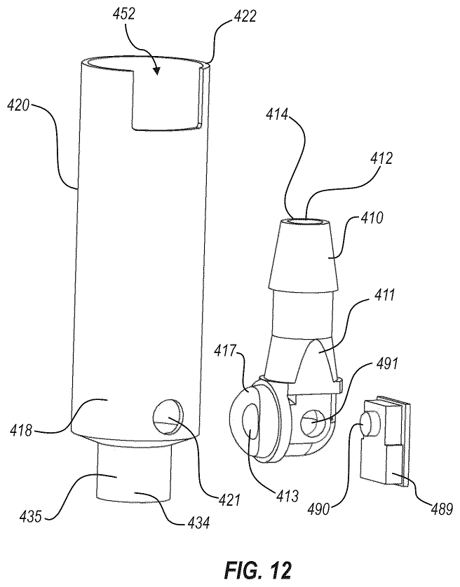

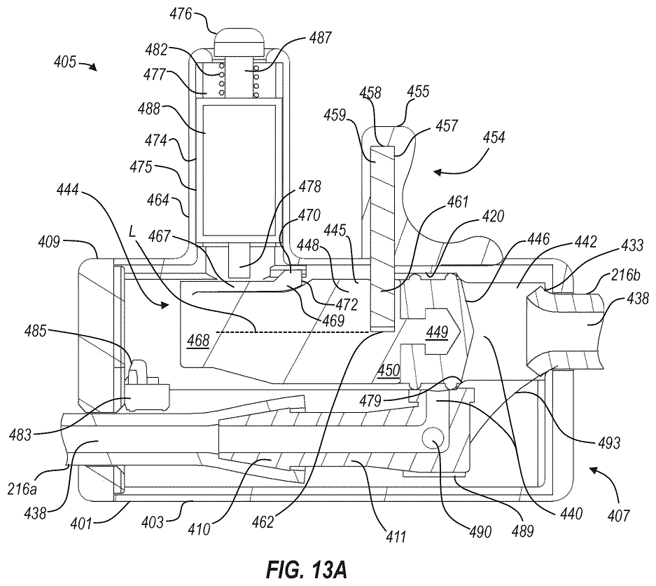

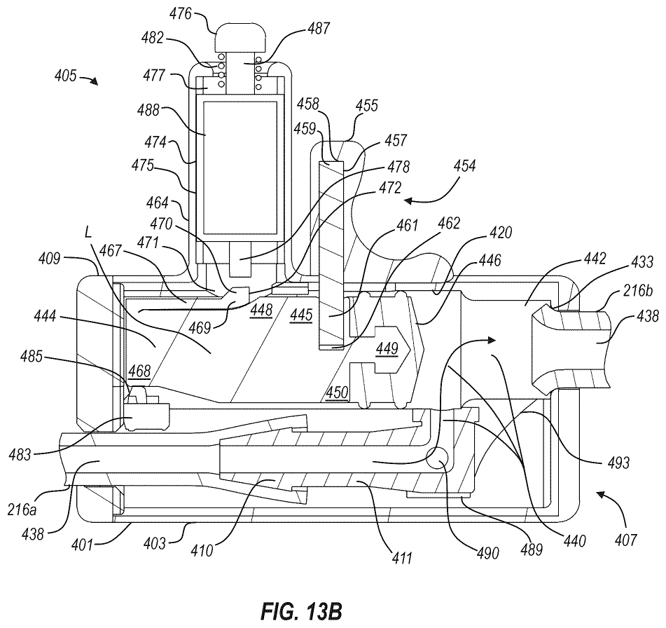

FIGS. 9-13B illustrate an aspiration tubing set 400 comprising sterile suction tubing 216 and the control 405. The control 405 may include a control body 407 having a housing 401 with a first housing half 402 and a second housing half 403, each configured to house several components, and to close on one another, and to attach to one another. The halves 402, 403 may be bonded together with adhesive, epoxy, or fused with ultrasonic welding or solvent welding, or may be secured together with screws or other connecting elements. As shown in FIG. 9, an aspiration passage 438 from the aspiration lumen 106 of the aspiration catheter 202 extends from left to right. A first connector 270 may be a luer fitting configured to sealingly attach to the luer 115. However, alternatively, the luer 115 may be replaced by a barb and the first connector 270 may be a suction connector, for example a 22 French silicone suction connector. The first connector 270 is sealingly secured to a first end 404 of a first sterile suction tubing 216a, which extends into an inlet 406 of the control body 407 at its second end 408. The inlet 406 comprises a hole within an end cover 409 that attaches to an end of the halves 402, 403. The second end 408 is frictionally slid over a barb 410 of an elbow fitting 411 of the control body 407, which includes an inner passage 412 having a 90° curve 415. In some embodiments, the curve 415 may comprise an acute angle, such as an angle between about 10° and about 80°, or between about 20° and about 70°, or between about 30° and about 60°, or a range having any two of the foregoing as endpoints. The inner passage 412 of the elbow fitting 411 has an inlet 413 and an outlet 414 (see FIG. 12). Surrounding the inlet 413 is a concave radiused surface 417 configured to be sealingly bonded to a convex cylindrical surface 418 on a custom syringe barrel (piston cylinder) 420. An entry orifice 421 passes through a wall 422 of the cylinder 420, starting at the surface 418. In some embodiments, the elbow fitting 411 and the cylinder 420 may be monolithic. For example, they may comprise a single injection-molded or 3D-printed component (or using other additive fabrication methods). A first end 433 of a second sterile suction tubing 216b is frictionally fit over an outer cylindrical surface 434 on a hub 435 of the cylinder 420. In some embodiments, an adhesive, epoxy, or welding may be used to seal the internal wall surface of the tubing 216b to the outer cylindrical surface 434, though the friction fit is sufficient for resisting-1 atmosphere of vacuum. In an alternative embodiment, the hub 435 includes a barb. In a further alternative embodiment, shown in FIGS. 13A-13B, the first end 433 may be flared and bonded and/or wedged between the halves 402, 403.

At a second end 436 of the tubing 216b, a second connector 261 is sealingly bonded. The second connector 261 may be a suction connector, for example a 30 French silicone suction connector. The second connector 261 may be configured to sealingly connect to a port of the canister 218, or to an intermediate tube that is then attached to the canister 218, or to the filter 220. Thus, from the first connector 270 to the second connector 261, a continuous, contained aspiration passage 438 is formed. As shown in FIG. 13B by an S-shaped arrow, the passage 438 comprises an S-duct 440 that is formed by the combination of the sterile suction tubing 216a, the elbow fitting 411, the cylinder 420, and the suction tubing 216b. The passage 438 may be configured for the transport of thrombus and blood from the aspiration lumen 106 of the aspiration catheter 202 to the canister 218.

Referring to FIGS. 13A-13B, the S-duct 440 provides the interior space of a valve 442, via the sliding of an occlusion member 444. The occlusion member 444 may comprise a piston structure 445 formed by a plunger 446 carried by a piston body 448. The occlusion member 444 may also comprise a plug, a rigid slider, a swinging separator, a rotary valve, or other structure configured to obstruct the passage 438. The occlusion member 444 (e.g., the piston body 448) may comprise a rigid polymer, such as a polyamide. FIG. 13A illustrates the valve 442 in a closed position, stopping flow through the passage 438, and FIG. 13B illustrates the valve 442 in an open position, allowing flow through the passage 438. FIGS. 13A and 13B illustrate an occlusion member 444 comprising a plunger 446 snapped onto a barb 449 at a first end 450 of the piston body 448, and is configured to move in unison with the piston body 448 within a cylindrical cavity 452 (FIG. 12) within the cylinder 420, the cylindrical cavity 452 having an internal volume. Alternatively, the plunger 446 may couple to the piston body 448 by screwing, clipping, friction fitting, or other means.

The control 405 may comprise a control interface 454 for moving the control between the closed position and the open position. The control interface 454 may comprise a slider 455, a lever, or a button. FIG. 13A illustrates a control interface 454 comprising a slider 455 that may include a concavity 456 for engaging the hand of a user and may also include a hole 457 into which a first end 458 of a spring pin 459 is frictionally engaged. Alternatively, the spring pin 459 may be bonded into the hole 457. The spring pin 459 is configured to be slidable, back-and-forth within an elongate slot 460 in the cylinder 420. The second end 461 of the spring pin 459 is frictionally engaged into a hole 462 in the piston body 448. The housing 401 includes a handle 464 extending transversely therefrom, and configured for holding or grasping (e.g., by the user's hand or one or more fingers) in opposition to the concavity 456 of the slider 455. Thus, by forcing the slider 455 to move relative to the housing 401 toward the handle 464, in the direction of the arrow 465 (FIG. 10) that is molded or otherwise marked on the side 466 of the first half 402, the piston body 448 is retracted, bringing the plunger 446 with it, and thus changing the valve 442 from the closed position of FIG. 13A to the open position of FIG. 13B.

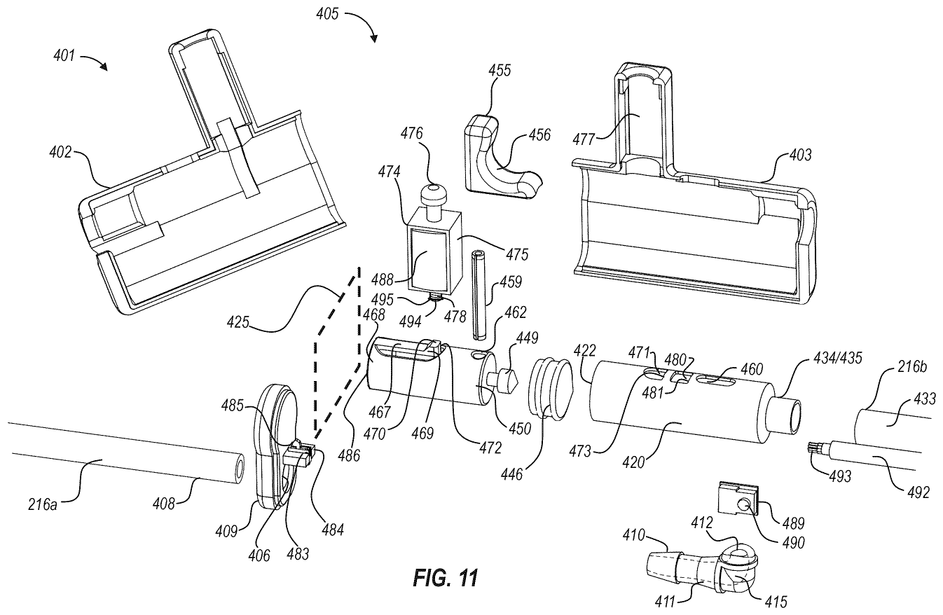

The piston body 448 further includes a cantilever beam 467 extending longitudinally from a second end 468 of the piston body 448 and terminating in a free beam end 469 having a locking tab 470. When the valve 442 is in its open position, the locking tab 470 is configured to extend out through a locking hole 471 in the wall 422 of the cylinder 420. Because the valve 442 is open, the negative pressure within the passage 438 sucks, and thus pulls, the plunger 446 (and thus, the piston body 448) in the opposite direction from the arrow 465 on the first half 402 of the housing 401. Thus, with features best seen in FIG. 11, an extreme edge 472 of the locking tab 470 stops against a distal ledge 473 of the locking hole 471, maintaining and locking the valve 442 in the open position. An unlocking assembly 474, comprising a casing 475 and a button 476, is slidable within a cavity 477 of the handle 464 in a direction generally transverse to longitudinal axis L of the cylinder 420/piston body 448. A user may voluntarily unlock the piston body 448 from the cylinder 420 by pressing the button 476 in direction D, causing an engagement pin 478 of the unlocking assembly 474 to push the locking tab 470 transversely inward (also generally in direction D) while causing the cantilever beam 467 to flex or deflect, thus moving the entirety of the locking tab 470 into the cylinder 420, fully free of the locking hole 471, and no longer engaged with the distal ledge 473. Therefore, the negative pressure within the passage 438 is now able to cause the plunger 446 (and thus, the piston body 448) to move in the opposite direction from the arrow 465 such that the valve 442 forcibly closes. The plunger 446 being sealingly forced against an interior annular edge 479 at the end of the cylinder 420. In this closed position, the locking tab 470 is moved to a position adjacent a resting hole 480 (FIG. 11), and the spring memory of the cantilever beam 467 causes the locking tab 470 to move transversely (generally opposite direction D), and through the resting hole 480. The negative pressure within the passage 438 forces the plunger 446/piston body 448, with some compression of the elastomeric plunger 446 material, such that the extreme edge 472 of the locking tab 470 is stopped against a resting ledge 481, thus freezing the piston body 448 in a single position with the valve 442 closed. Flow through the passage 438 is thus interrupted. A compression spring 482 may be carried on a shaft 487 below the button 476 to cause the button 476 and the unlocking assembly 474 to return to the original position after the button 476 is depressed and then released.

An electrical switch 483 may be carried within a groove 484 of the end cover 409 and includes a spring-loaded, displaceable switch button 485. When the valve 442 is opened, the piston body 448 is slid toward the switch 483 such that an annular edge 486 of the second end 468 of the piston body 448 engages and moves the switch button 485, thus activating the electrical switch 483. In some embodiments, the activation of the electrical switch 483 causes the pump of the SDU 212 to start injecting pressurized fluid through the supply lumen 114 of the catheter 202. In some embodiments, the deactivation of the electrical switch 483 causes the pump of the SDU 212 to stop. The movement of the piston body toward the direction in which the valve 442 is closed causes the annular edge 486 to move away from and stop engaging the switch button 485, thus shutting off the switch. In some embodiments, the electrical switch 483 may comprise an SPST-NO switch. Thus, the opening and closing of the valve 442 and the turning on and off of the pump of the SDU 212 are synchronized together by a combination electric and hydraulic switch comprised by the control 405. In manual operation, a user opens the valve 442 and also turns on the pump of the SDU 212 by moving the slider 455. The user then closes the valve 442 and turns off the pump of the SDU 212 by pushing the button 476. The controller 235 is configured to receive the signal from the switch 483, and to turn the pump of the SDU 212 to start (or stop) immediately, or with a particular delay time.

An emergency shut-off is provided by a solenoid 488 within the unlocking assembly 474, and a pressure sensor 489 configured to measure the pressure within the passage 438. Turning to FIG. 12, the pressure sensor 489 includes a sensing portion 490 which is inserted through a hole 491 in the elbow fitting 411. The surrounding portions of the pressure sensor 489 and the elbow fitting 411 are then sealed with epoxy, adhesive, or other means. Thus, the pressure sensor 489 is capable of measuring the pressure within inner passage 412 of the elbow fitting 411 and outputting a signal related to the measured parameter. Alternatively, the pressure sensor 489 (or another pressure sensor) can be configured to measure the pressure in another location along the passage 438, or even in the aspiration lumen 106 of the catheter 202. A wire conduit 492 extends in parallel along the second sterile suction tubing 216b and carries one or more or two or more conductors 493. The conductors 493 may each comprise insulated copper wire. The conductors 493 are configured to deliver power and/or to carry signals to and from the pressure sensor 489, the switch 483, and the solenoid 488, as well as the additional pressure sensor 230, if used. As mentioned, at the second end 436 of the tubing 216b, the conductors 493 terminate via electrical connection to a modular plug connector 236 that is configured to snap into the mating receptacle 237 in the SDU 212 (FIGS. 4-5). The connector 236 may in some embodiments be an eight position, eight contact (8p8c) connector.

While the valve 442 is in the open position and the pump of the SDU 212 is operating (e.g., via the saddle 283), a malfunction may occur that causes a loss in negative pressure within the passage 438. The user may not realize that this has happened, and thus the potentially hazardous situation of injecting the pressurized fluid without any aspiration may occur. The pressure sensor 489 inputs to the controller 235, which is configured to recognize when an unacceptable pressure is being read (e.g., insufficient level of vacuum). The controller 235 is configured to temporarily energize the solenoid 488, which causes the engagement pin 478 of the unlocking assembly 474 to telescopically extend from the solenoid 488, thus electromagnetically energizing the solenoid 488 to move the locking tab 470 transversely inward (direction D) while causing the cantilever beam 467 to flex, pushing the entirety of the locking tab 470 within the cylinder 420, free of the locking hole 471. This allows the valve 442 to close and the pump of the SDU 212 to be immediately shut off, avoiding the potentially hazardous situation. A spring 494 and retaining ring 495 are shown in FIG. 11 and are configured to return the engagement pin 478 to its un-extended position, after the controller 235 stops energizing the solenoid 488. Other safety features related to system shut-down, or other automatic system responses, may be utilized, such as those described in U.S. Pat. No. 10,716,583, issued Jul. 21, 2020, and entitled, “Systems and Methods for Removal of Blood and Thrombotic Material”, U.S. Pat. No. 10,492,805, issued Dec. 3, 2019, and entitled, “Systems and Methods for Thrombosis and Delivery of an Agent” or U.S. Pat. App. Pub. No. 2018/0207397, published Jul. 26, 2018, and entitled, “Systems and Methods for Removal of Blood and Thrombotic Material”, all of which are incorporated by reference in their entirety for all purposes.

Returning to FIG. 5, a solenoid 298 is carried internally in the SDU 212, and is configured to interface with the interior 296 of the canister 218, via the suction tubing 214, or via any additional tubing. The solenoid 298 is configured to vent the negative pressure inside the canister 218, by opening a valve 299 coupled to the solenoid (mechanically or electromagnetically) that opens the interior 296 of the canister 218 to ambient pressure. The venting allows any foaming of blood or fluid, such as any aspirated liquid, within the canister 218 to be reduced. Foaming can occur during a thrombolysis procedure due to cavitation, as air bubbles are formed. The solenoid 298 is then configured to close the valve 299, to allow negative pressure to again be built up within the interior 296 of the canister 218. The controller 235 is configured to automatically energize the solenoid 298, in order to allow for the degassing/defoaming. For example, the controller 235 may send a signal to energize the solenoid 298 based on the measurement of a targeted negative pressure and/or a targeted time of aspiration cycle. In other cases, the controller 235 can send a signal to energize the solenoid 298 every minute, every five minutes, every ten minutes, etc. Additionally, a user can operate the controller 235, and more generally the controller 174, of the system 200 through the interface panel 290 to initiate degassing/defoaming of the interior 296. The venting may also be able to remove air bubbles inside the other lumens of the catheter and tubing sets.

In some embodiments, the controller 235 can output or send a signal to energize the solenoid 298 to open the valve 299, in order to stop any aspiration, while still allowing the SDU 212 to deliver saline, medication, or saline combined with medication (e.g., thrombolytic drugs), so that the fluids can be delivered out of the open distal end 107 (instead of being aspirated through the aspiration lumen 106).

The control 405 may suffer from a potential flaw in an incompletely-closing valve 442 as a result of manufacturing imperfections and/or erosion from extended use. This may result in application of vacuum to the aspiration lumen 106 at inopportune moments which may present an opportunity for harm to the patient. For example, an incompletely-closing valve 442 may apply vacuum to the aspiration lumen 106 when the pump 101 is deactivated (e.g., when the pump 101 is intentionally turned off or during loss of power), which may detrimentally remove excess blood from the patient. This flaw may arise, for example, from imperfections in the components of the control 405 generated during the manufacturing and assembly processes or generated from wear experienced through prolonged use of the device.

For example, in some cases, the valve 442 of control 405 may fail to completely close despite the plunger 446 being completely positioned in the valve 442 because of small tears and/or gaps in the plunger surface. In other cases, friction between the piston structure 445 and cylinder 420 may prevent the plunger 446 from being positioned completely in the valve 442.

Additional embodiments of the control are presented including additional components and/or control designs to remedy this potential flaw. The additional components and/or control designs may comprise mechanical and/or electrical mechanisms. Also, additional components and/or control designs comprising relatively simple mechanisms may be advantageous in that they may generally provide more robust mechanisms for completely closing the valve 442. The additional components and/or control designs may also improve the ergonomics of the control by promoting the case with which the control may be held.

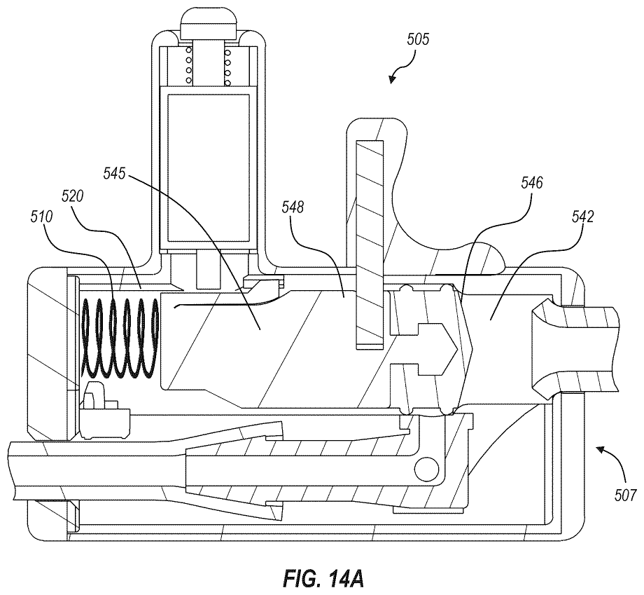

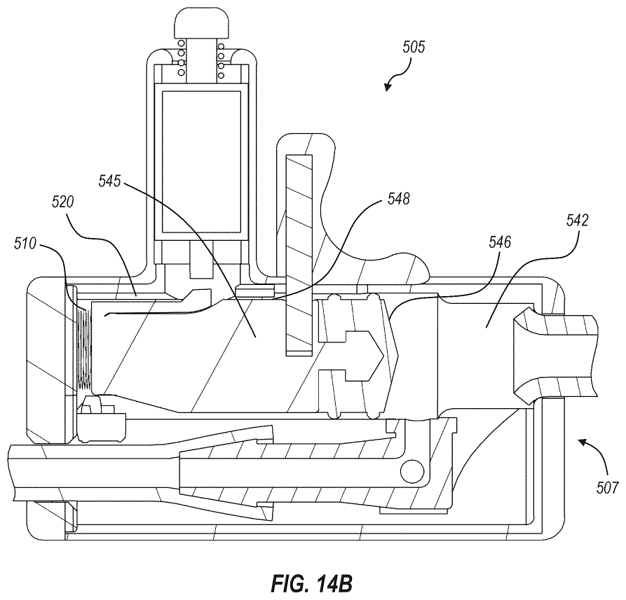

FIGS. 14A and 14B illustrate an exemplary embodiment of a control 505, wherein the control 505 may include a return mechanism comprising a return spring 510 positioned proximally of the piston structure 545 within the cylinder 520 of the control body 507. The return mechanism may be located at a position 425 (FIG. 11) proximal of the piston structure 545, but may alternatively be positioned between the plunger 546 and the piston body 548. The return mechanism may comprise mechanical means, such as a spring (e.g., compression spring, extension spring, torsion spring, wave disk spring, volute spring, spiral spring, leaf spring, etc.), or electrical means, such as a solenoid. The return mechanism functions to push the piston structure, including the plunger, into the valve 542 and thereby completely close the valve 542 (as seen in FIG. 14A). Imperfections that may be present in the plunger surface (e.g., wear-induced tears and gaps resulting from prolonged used of the control 505) may closed from a force exerted by the return mechanism on the piston structure 545 and/or plunger 546. The return mechanism may also be configured to exert a force on the piston structure 545 and/or plunger 546 when the control 505 is moved from an open to a closed position, and thereby aid the plunger 546 to move into the valve 542.

The return spring 510 may comprise a compression spring and may be configured in a compressed state within the control 505. FIG. 14B illustrates that the return spring 510 may be placed in a high-compression state when the control 505 is moved to the open position. The return spring 510 may also be placed in a low-compression state by moving the control 505 into the closed position. While in the closed position, the force of the return spring 510 while in the low-compression state enables the return spring 510 to exert a force upon the plunger 546 or piston structure 545 sufficient to facilitate complete closing of the valve 542 by the plunger 546. Although in the closed position of the control 505 (FIG. 14A) the return spring 510 is placed in a low-compression state, the force exerted by the return spring 510 while in the low-compression state may be sufficient to provide significant force to the plunger 546 or piston structure 545 to enable complete closure of the valve 542 by the plunger 546. The return spring 510 may also be particularly useful in moving the piston body 548 distally along the cylinder 520 upon deactivation of the pump 101 or during an emergency shut-off. This ensures that upon deactivation (whether intended or unintended) of the pump 101 excess blood is not evacuated from the patient. Inclusion of the return spring 510 in the control 505 of the above configuration may support a plunger 546 in completely closing the valve 542 in instances where the plunger 546 deteriorates over extended use or where the plunger 546 may suffer from imperfections of the manufacturing process.

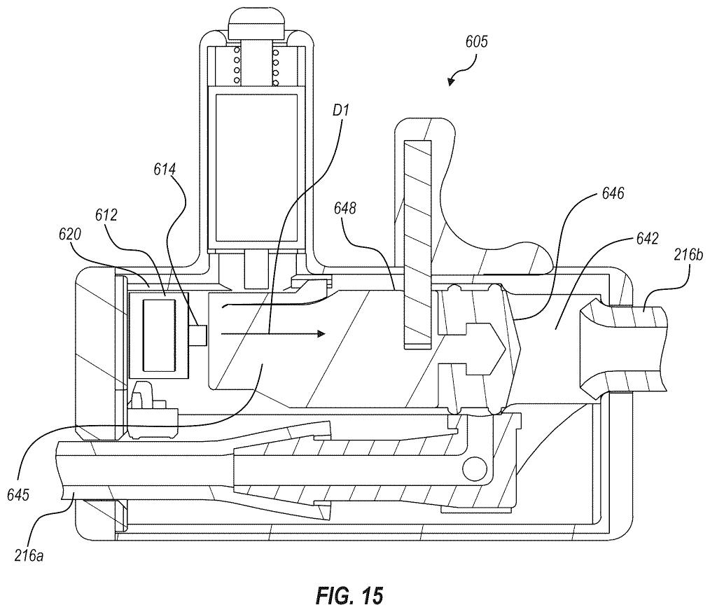

FIG. 15 illustrates another embodiment of a control 605, including a return mechanism comprising a solenoid 612 that may be positioned proximally of the plunger 646 or piston body 648 in a manner similar to the return spring 510 described above, and which may provide the similar function the return spring 510 of maintaining the valve 642 completely closed while the control 605 is in the closed position. Additional components comprising solenoids may be beneficial because they may be incorporated into fast switching valves and have generally good reliability and long service lives. The solenoid 612 may be configured to activate upon unlocking of the piston body 648, deactivation of the pump 101, and/or during an emergency shut-off, during which the solenoid may push an engagement pin 614 distally in a direction D1 against the piston body 648 or plunger 646 to push the plunger 646 into and completely close the valve 642. A solenoid 612 so configured may provide benefits similar to the return spring 510 described above by helping to ensure that the piston structure 645 travels along the cylinder 620 and that the valve 642 remains completely closed when the control 605 is in the closed position and thus preventing excess blood from being evacuated from the patient.

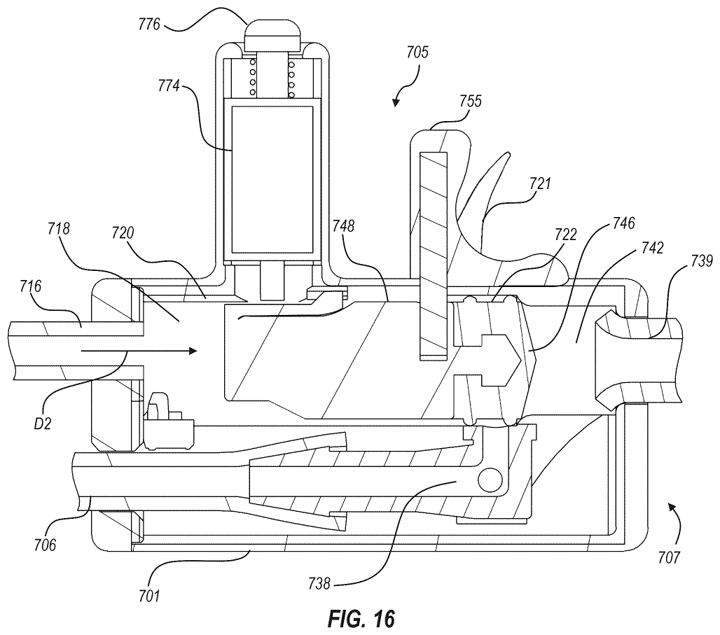

FIG. 16 illustrates a control 705 wherein the return mechanism comprises pressurized air, such that the valve 742 comprises a pneumatic valve. Pressurized air may be delivered to the control 705 via a pneumatic line 716 attached to a pressure chamber 718 contained within the cylinder 720 of the control body 707 and positioned proximally of the plunger 746. The pneumatic line 716 may be attached to at least a portion of the sterile suction tubing 216a (as seen in FIG. 9) to enable easy maneuvering of the sterile suction tubing 216 and control 705. Moving the control 705 from the open position to the closed position (e.g., by pressing the button 776 of the unlocking assembly 774) may cause the SDU 212 to activate an air pump to pressurize the pneumatic line 716 and the pressure chamber 718 of the cylinder 720 connected thereto. Alternatively, moving the control 705 from the open position to the closed position may cause open a connection from an air pressure source to the pneumatic line 716 and pressure chamber 718, such that the pneumatic line 716 and pressure chamber 718 are pressurized. The surface 722 of the plunger 746 may be formed tightly against the cylinder 720 so as to isolate the aspiration passage 738 of the valve 742 from the pressure chamber 718 of the cylinder 720 and prevent pressurized air from entering the aspiration lumen 106 When the control 705 is released from the open position (e.g., when the solenoid pushes the locking tab 470 out), pressurized air may pass through the pneumatic line 716 into the pressure chamber 718 and push the piston body 748 and plunger 746 in a distally direction D2 into, and thereby close, the valve 742.

A seal may be formed between the first and second halves of the housing 701, the housing 701 having sufficient strength to withstand the pressure provided by the pneumatic line 716, such that the pressure chamber 718 remains pressurized. The control 705 may include a venting actuator 721 configured to de-pressurize the pressure chamber 718. The venting actuator 721 may comprise a trigger (as shown in FIG. 16), button, lever, or other actuator. The venting actuator 721 may be attached to the exterior of the control housing 701, but may preferably be attached to the slider 755 (as shown in FIG. 16), enabling a user to easily de-pressurize the pressure chamber 718 and pull the slider 755 to the open position in the same motion.

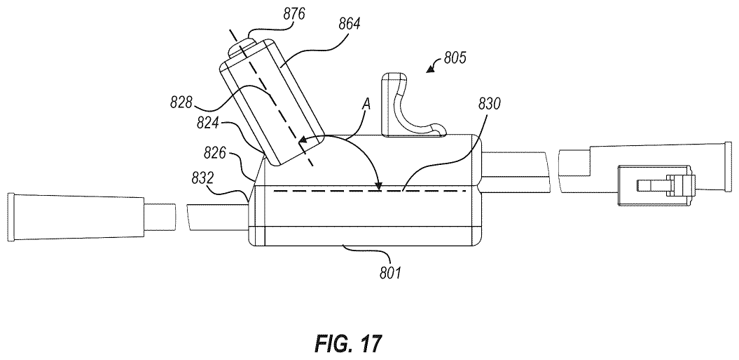

The control body 707 may be relatively long (e.g., 6 to 10 inches as measured from the inlet 706 to the outlet 739 of the control body 707) so as to enable the control 705 to be more easily gripped by the user. FIG. 17 illustrates a control 805 that enables improved gripping of the control 805 by positioning the longitudinal axis 828 of the handle 864 at an oblique angle A relative to a longitudinal axis 830 of the housing 801. The handle 864 may be positioned at the oblique angle A within a range of approximately 45° to approximately 170°, or approximately 70° to approximately 150°, or approximately 90° to approximately 135°, or a range with any two of the foregoing as endpoints. An unlocking assembly, which may include similar features to unlocking assembly 474 described above, may be similarly located within the handle 864, including a button 876 configured to activate a solenoid configured for unlocking the piston structure. The handle 864 may be positioned at or near a distal end 832 of the control 805. The control 805 may also include a notch 824 (i.e., a distally-extending recess of the housing 801) configured to receive the purlicue of a hand of the user, the notch 824 being formed at the intersection of the handle 864 and a proximally extending portion 826 of housing 801. The notch 824 may thus provide a comfortable grip for the user and prevent the hand of the user from sliding along the handle 864.

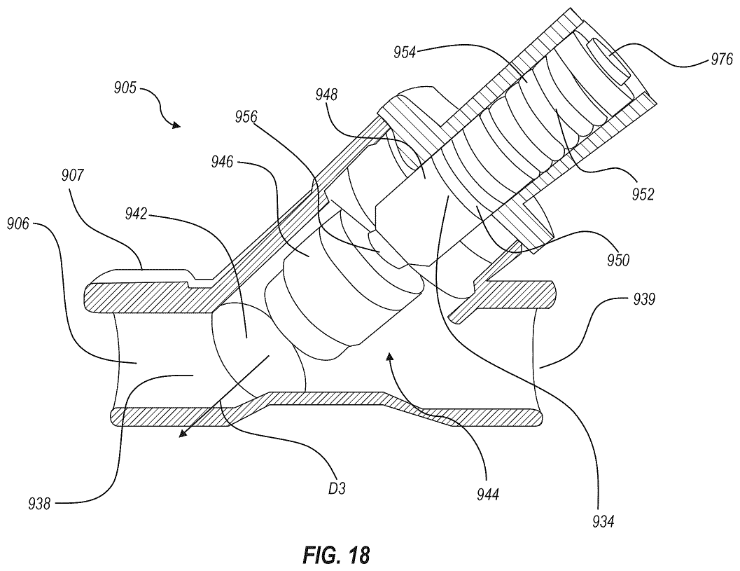

FIG. 18 illustrates another control 905 comprising a control body 907, having an aspiration passage 938 connected by an inlet 906 and an outlet 939 disposed therein, the aspiration passage 938 connecting the aspiration lumen 106 of the catheter 102 to the vacuum source, and a valve 942 disposed at least partially within the aspiration passage 938. The control may also include an occlusion member 944 comprising a piston body 948 and a plunger 946 attached to the distal end 934 of the piston body 948. The control 905 may be moved into a closed position by positioning the plunger 946 into the valve 942 and blocking the flow of aspirant through the valve 942 in a manner similar to that described in other embodiments above. The control 905 may also include a button 976, a solenoid 950, a pin 952 disposed within the solenoid 950 (which may be attached or affixed to the proximal end 956 of the piston body 948), and a spring 954, the button 976, solenoid 950, and spring 954 configured to move the control 905 between an open and closed position. The spring 954 may comprise a compression spring, extension spring, wave-ring spring, or other spring, similar to spring 510 described above. The spring 954 may be disposed between the solenoid 950 and the button 976 and attached to the distal end of the pin 952. The solenoid 950 may be configured to have a polarity when the control 905 is in the open position and no polarity when the control 905 is in the closed position. Alternatively, the solenoid 950 may be configured to have no polarity when the control 905 is in the open position and a polarity when the control 905 is in the closed position. Pressing the button 976 may alter the polarity of the solenoid 950. Thus, the control 905 may be moved from the open position to the closed position, or from the closed position to the open position, by pressing the button 976.

For example, the control 905 may be moved from the open position to the closed position by pressing the button 976 which creates a polarity over the solenoid 950 and forces the pin 952 (and thus the piston body 948 and plunger 946 attached thereto) in a substantially distal direction D3 and closing the valve 942. This action may also extend the spring 954, such that the spring 954 is placed in tension. Pressing the button again removes the polarity from across the solenoid 950 such that a force in the distal direction D3 is no longer applied to the pin 952. The spring 954 then acts to pull the pin 952 (and the piston body 948 and the plunger 946 attached thereto) in a substantially proximal direction opposite the substantially distal direction D3, reopening the valve 942.

In another embodiment, the function of the solenoid 950 and spring 954 may be reversed, the solenoid 950 acting to keep the control 905 in an open position and the spring 954 acting to push the plunger 946 into the valve 942 and move the control 905 into a closed position. For example, while the control 905 is in the open position a polarity may be provided across the solenoid 950 such that the pin 952 is pushed in a substantially proximal direction opposite the substantially distal direction D3 and the spring 954 is compressed, maintaining the plunger 946 out of the valve 942. By pressing the button 976, the solenoid may be deactivated and allow the spring 954 to push the pin 952, and the piston body 948 and plunger 946 attached thereto, in the substantially distal direction D3, such that the plunger 946 closes the valve 942. Pushing the button 976 again may activate the solenoid 950, pushing the pin 952 proximally, and the plunger 946 and piston body 948 attached thereto, to the open position and re-compressing the spring 954.

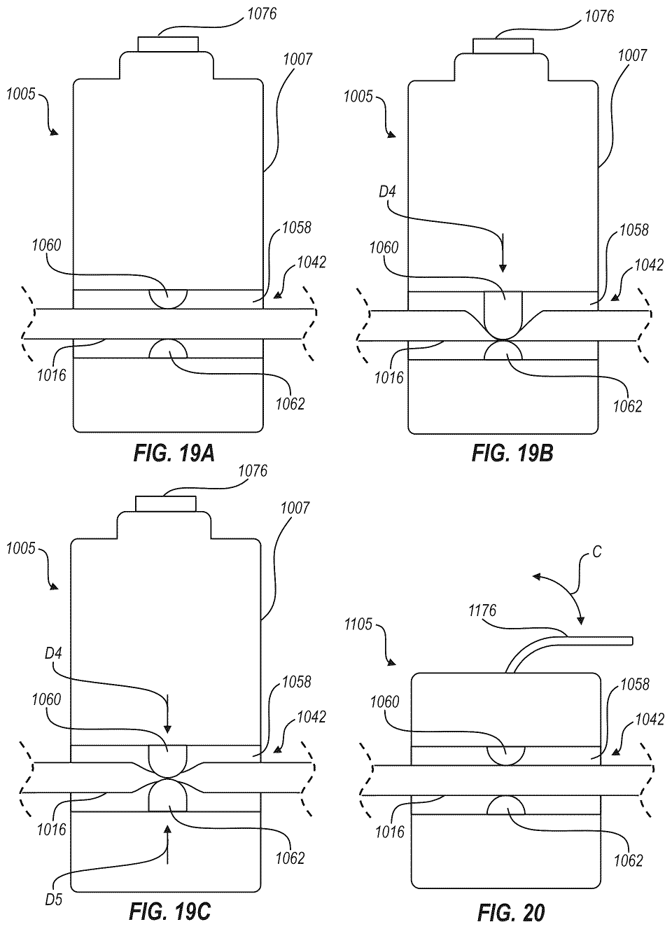

The systems 100 or 200 may alternatively employ a control having a different type of valve other than valves employing plungers or plugs. For example, the valve of the control may comprise a rotary valve, a pinch valve, or other valve. FIGS. 19A-19C illustrate a control 1005 having a pinch valve 1042. Sterile suction tubing 1016, connecting the aspiration lumen 106 with a vacuum source, may be threaded between a first and second anvil 1060, 1062 positioned to either side of a channel 1058 disposed within the control body 1007, the channel and first and second anvils 1060, 1062 forming a pinch valve 1042. A pinch valve 1042 may beneficially be employed in conjunction with smaller tubing 1016 than that used in the electronic valves described above, which may aid in reducing blood loss from the patient. The pinch valve 1042 may also be used with tubing 1016 of various diameters, making the control 1005 more procedurally flexible. The head of the first and/or second anvils 1060, 1062 may have a curved or circular surface, but may alternatively have a head with a pointed surface or a flat surface. An activation button 1076 may be connected to the control 1005 to activate the pinch valve 1042. FIG. 19A illustrates the pinch valve 1042 in the open position. Upon activation of the pinch valve 1042, the pinch valve 1042 may be moved to the closed position wherein, as illustrated in FIG. 19B, the first anvil 1060 may move in a first direction D4 towards the second anvil 1062 such that the distance between anvils 1060, 1062 decreases and the anvils 1060, 1062 close or pinch the sterile suction tubing 1016, disconnecting the aspiration lumen 106 from the vacuum source. Pressing the activation button 1076 again may move the first anvil 1060 in a second direction opposite the first direction D4 to reopen the sterile suction tubing 1016 and connect the aspiration lumen 106 to the vacuum source. Activation of the pinch valve 1042 may also cause both the first and second anvils 1060, 1062 to move into the channel 1058, as illustrated in FIG. 19C. In this instance, the first anvil 1060 may move in a first direction D4, while the second anvil 1062 may move in a second direction D3 opposite the direction of D4, the first and second anvils 1060, 1062 coming together to close the valve 1042. Pressing the activation button 1076 again may cause both the first and second anvils 1060, 1062 to open the valve 1042 by moving in opposite directions than when the pinch valve 1042 was first activated.

FIG. 20 illustrates a control 1105 having a pinch valve 1042 wherein the valve 1042 is manually actuated. For example, the control 1105 may comprise a lever 1176 that may rotate along curve C to open and close the valve 1042 in a manner similar to the pinch valves of FIGS. 19A-19C described above. A manually actuated valve, including the manually actuated pinch valve 1042 of control 1105, may provide a benefit over the automatically and/or electronically actuated controls by providing tactile feedback to the user and aiding the user to know that the valve 1042 is closed without having to look at the valve 1042 directly. Manually actuated valves are also beneficial in that they are generally smaller, and thus less cumbersome, than automatically and/or electronically actuated valves.

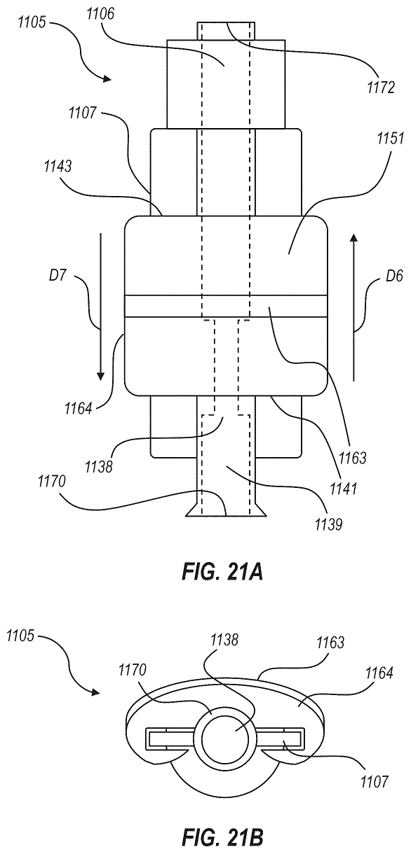

The control may also comprise additional and/or other mechanical components that may not rely on electrical components to open and close the vacuum line. In particular, the control may comprise a manual actuation. FIGS. 21A-21B illustrate a control 1105 comprising a slider switch 1164. FIG. 21A illustrates a plan view of the control 1105, the control 1105 having a control body 1107 comprising an aspiration passage 1138 (indicated by the dotted line) connected by an inlet 1106 and an outlet 1139. The control 1105 also comprises a slider switch 1164 configured to obstruct the aspiration passage 1138 when placed in a first position. For example, the control 1105 may be placed in a closed position by moving the switch 1164 in a first direction D6 from which the control 1105 may be placed in an open position by moving the switch 1164 in a second direction D7 opposite that of the first direction D6. The movement of the switch 1164 in a first direction D6 may be a direction towards the vacuum source and away from the aspiration catheter 102, such that when the switch 1164 is placed against the opening of the sterile suction tubing 216b the negative pressure of the vacuum source pulls the switch 1164 up against an opening to the distal end 1172 of the control 1105, keeping the switch 1164 secured in the closed position. The control 1105 may include connection means (e.g., luer locks) at the proximal and distal ends 1170, 1172 for connecting the control 1105 to sterile suction tubing 216a, 216b. The connection means may be color coded (e.g., red at the proximal end 1170 and blue at the distal end 1172) to facilitate quick connection between the control 1105 and the sterile suction tubing 216a, 216b. The slider switch 1164 of the control 1105 may also comprise a tactile surface. For example, the slider switch 1164 may comprise a ridge 1163 protruding from an upper surface 1151 of the slider switch 1164. Alternatively, the upper surface 1151 of the slider switch may comprise a textured surface (e.g., a rough or bumpy surface) or an upper surface 1151 that peaks at or near the proximal 1141 and distal 1143 ends of the slider switch 1164.

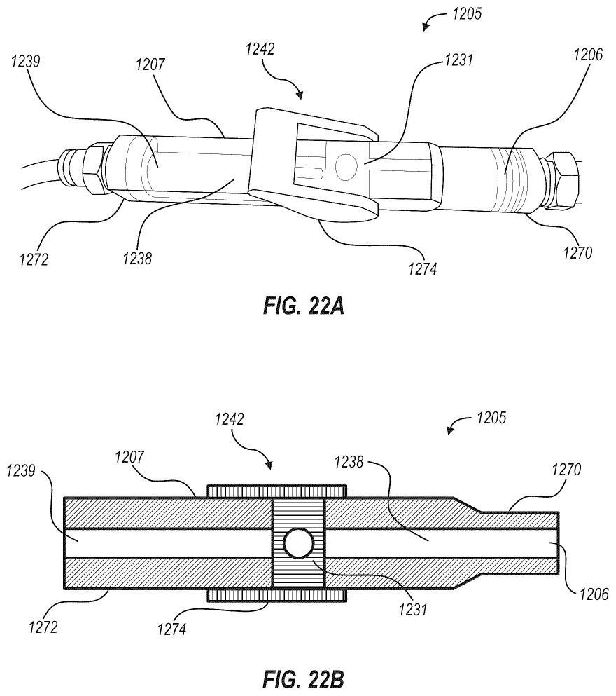

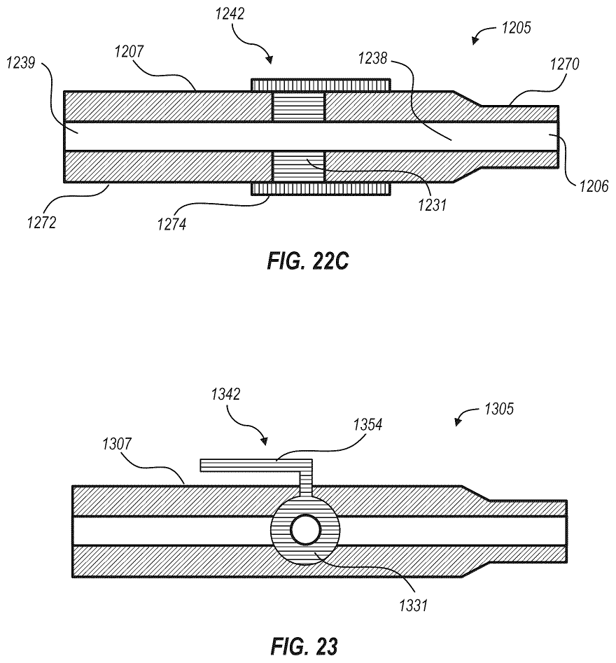

FIGS. 22A-22C illustrate a control 1205 comprising a rotary valve 1242. The control 1205 may comprise a control body 1207 having an aspiration passage 1238 disposed therein, the aspiration passage being connected by an inlet 1206 and an outlet 1239. As shown in FIG. 22A, the control body 1207 of the control 1205 may be cylindrical, but may comprise other shapes, such as a control body 1207 having a square, rectangular, triangular cross section, or having a cross section having another shape. The control body 1207 may be relatively long (e.g., 6 to 10 inches in length) and have a relatively small diameter (e.g., 0.5 to 2 inches in diameter) to facilitate gripping the control 1205 by the user. FIGS. 22B and 22C illustrate cross-sectional views of control 1205, showing that an aspiration passage 1238 may extend through the control 1205 connecting the aspiration lumen 106 of the aspiration catheter 102 with the vacuum source. A rotary valve may be at least partially disposed within the aspiration passage 1238. The rotary valve may comprise a stopcock 1231 (as shown in FIGS. 22A-22C), a ball valve (such as a quarter-turn ball valve 1331 connected to a control interface 1354 comprising a handle incorporated into the control 1305 shown in FIG. 23), or other rotating valve. The control 1205 may additionally, or alternatively, comprise other types of valves, such as linear valves, including gate valves and globe valves. A rotary valve 1242 may be located at or near the proximal or distal ends 1270, 1272 of the control 1205 for opening or closing of the aspiration passage 1238. The valve 1242 may have a range of 90° of rotation between completely opening and closing the valve 1242, but may comprise a valve having another range of rotation between completely opening and closing the valve 1242, such as a valve having 180° of rotation, or a valve having 360° of rotation. Specifically, valve 1242 may have a range of rotation between 0° and 90°, or between 0° and 180°, or between 225° and 360°. A handle 1274 may be attached to the valve 1242 to facilitate opening and closing of the aspiration passage 1238.

FIG. 23 illustrates a cross-sectional view of a similar control 1305, the control comprising a ball valve 1331 disposed at least partially within the aspiration passage 1138 of the control body 1307. The ball valve 1331 may offer an advantage over the stopcock 1231 of FIGS. 22A-22C in that a ball valve 1331 may withstand greater operating pressures than a stopcock 1231. The controls 1205 and 1305 may also comprise connections disposed at the proximal and/or distal ends 1270, 1272 for connecting the sterile suction tubing 216a, 216b to the controls 1205 and 1305. The connections may comprise connections for quickly connecting the sterile suction tubing 216a, 216b to the controls 1205, 1305, such as luer lock fittings, push-to-connect fittings, a barb configured to frictionally secure tubing disposed thereon, and/or quick-disconnect hose couplings. A control, such as controls 1205 and 1305, having a manual valve, such as the rotary valves 1242 or 1342 described above, may provide particular advantages over other designs in that they generally have greater durability, are intuitive and thus easier to train a user on how to operate, and are easier to source.

The controls describe herein may meet certain performance characteristics. For instance, when removing liquids with a similar viscosity profile as blood and/or clots using a 0.026″ jet location of an aspiration catheter, the controls may not leak. The controls may remove about 90 ml of saline in between about 20-25 seconds, about 20 ml of saline in about 13 seconds, about 90 ml of bovine blood in between about 55-80 seconds, about 20 ml of bovine blood in about 13 seconds, and/or between about 10-11 g of a synthetic blood clot in between about 30-55 seconds and/or between about 115-125 seconds depending on the aspiration catheter configuration and a sub-acute or medium toughness of the synthetic blood clot measured by shear and compression data.

Although the systems for aspirating thrombus described herein are predominantly focused on aspiration, the systems may also, or alternatively, be configured for injecting or infusing fluids, with or without drugs, and may incorporate related features described in U.S. Pat. No. 10,716,583, issued Jul. 21, 2020, and entitled, “Systems and Methods for Removal of Blood and Thrombotic Material” and U.S. Pat. No. 10,492,805, issued Dec. 3, 2019, and entitled, “Systems and Methods for Thrombosis and Delivery of an Agent”.

It is contemplated that various combinations or sub-combinations of the specific features and aspects of the embodiments disclosed above may be made and still fall within one or more of the embodiments. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed embodiments. Thus, it is intended that the scope of the present disclosure herein disclosed should not be limited by the particular disclosed embodiments described above. Moreover, while the present disclosure is susceptible to various modifications, and alternative forms, specific examples thereof have been shown in the drawings and are herein described in detail. It should be understood, however, that the present disclosure is not to be limited to the particular forms or methods disclosed, but to the contrary, the present disclosure is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately”, “about”, and “substantially” as used herein include the recited numbers (e.g., about 10%=10%), and also represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

For purposes of the present disclosure and appended claims, the conjunction “or” is to be construed inclusively (e.g., “an apple or an orange” would be interpreted as “an apple, or an orange, or both”; e.g., “an apple, an orange, or an avocado” would be interpreted as “an apple, or an orange, or an avocado, or any two, or all three”), unless: (i) it is explicitly stated otherwise, e.g., by use of “either . . . or,” “only one of,” or similar language; or (ii) two or more of the listed alternatives are mutually exclusive within the particular context, in which case “or” would encompass only those combinations involving non-mutually-exclusive alternatives. For purposes of the present disclosure and appended claims, the words “comprising,” “including,” “having,” and variants thereof, wherever they appear, shall be construed as open-ended terminology, with the same meaning as if the phrase “at least” were appended after each instance thereof.

Following are some further example embodiments of the invention. These are presented only by way of example and are not intended to limit the scope of the invention in any way. Further, any example embodiment can be combined with one or more of the example embodiments.

Embodiment 1. A control configured for use in a system for aspirating thrombus, the control having an open position and a closed position, the control comprising a control body having an inlet and an outlet connected by an aspiration passage, a valve disposed at least partially within the aspiration passage, an occlusion member disposed at least partially within the control body, wherein the occlusion member is configured to enter the valve and obstruct flow of a fluid through the aspiration passage, a control interface configured to be manipulated by a user to move the control between the open and closed positions, and a return mechanism configured to facilitate movement of the occlusion member into the valve.

Embodiment 2. The control of embodiment 1, wherein the occlusion member applies a force to the occlusion member to maintain the valve in the closed position.

Embodiment 3. The control of embodiment 1 or 2, wherein the return mechanism is disposed between the occlusion member and the control body.

Embodiment 4. The control of any one of embodiments 1 through 3, wherein the return mechanism is adjacent a first end of the occlusion member opposite a second end of the occlusion member that enters the valve.

Embodiment 5. The control of any one of embodiments 1 through 4, wherein the return mechanism comprises a return spring.

Embodiment 6. The control of embodiment 5, wherein the return spring comprises a compression spring.

Embodiment 7. The control of embodiment 5, wherein the return spring comprises an extension spring.

Embodiment 8. The control of embodiment 5, wherein the return spring comprises a compression spring, an extension spring, a torsion spring, a wave disk spring, a volute spring, a spiral spring, or leaf spring.

Embodiment 9. The control of any one of embodiments 1 through 4, wherein the return mechanism comprises a solenoid.

Embodiment 10. The control of embodiment 9, further comprising an engagement pin disposed within the solenoid.

Embodiment 11. The control of embodiment 10, wherein the engagement pin is configured to push the occlusion member into the valve upon activation of the solenoid.

Embodiment 12. The control of embodiment 10, wherein the engagement pin is affixed to the occlusion member.

Embodiment 13. The control of embodiment 10, wherein the solenoid is affixed to the occlusion member.

Embodiment 14. The control of any one of embodiments 1 through 4, wherein the return mechanism comprises a pneumatic connection.

Embodiment 15. The control of embodiment 14, wherein the control body comprises a pressure chamber, wherein the pressure chamber is adjacent a first end of the occlusion member opposite a second end of the occlusion member that is configured to enter the valve.

Embodiment 16. The control of embodiment 14 or 15, wherein the control interface comprises a venting actuator.

Embodiment 17. The control of any one of embodiments 9 through 16, further comprising a spring configured to move the occlusion member out of the valve.

Embodiment 18. The control of any one of embodiments 1 through 17, wherein the control interface comprises a slider.

Embodiment 19. The control of any one of embodiments 1 through 17, wherein the control interface comprises a button.

Embodiment 20. The control of any one of embodiments 1 through 19, further comprising an electrical switch configured to be activated by the occlusion member when the occlusion member is moved to the open position.

Embodiment 21. The control of embodiment 20, wherein the electrical switch is configured to activate a vacuum source.

Embodiment 22. A control configured for use in a system for aspirating thrombus, the control having an open position and a closed position, the control comprising a control body having a longitudinal axis and configured to be moved between an open position and a closed position and having an aspiration passage, a handle having a longitudinal axis transverse to the longitudinal axis of the control body, a control interface configured to be manipulated by a user to move the control body between the open position and the closed position, a valve disposed at least partially within the aspiration passage, wherein when the control is in the closed position the valve is configured to obstruct flow of a fluid through the aspiration passage, and wherein when the control is in the open position the valve is configured to allow flow through the aspiration passage.

Embodiment 23. The control of embodiment 22, wherein an angle between the longitudinal axis of the handle and the longitudinal axis of the control body is within a range of approximately 45° to approximately 170°, or approximately 70° to approximately 150°, or approximately 90° to approximately 135°, or a range with any two of the foregoing as endpoints.

Embodiment 24. The control of embodiment 22, wherein the control body comprises a notch adjacent the handle configured to receive a purlicue of a hand of a user.

Embodiment 25. A control configured for use in a system for aspirating thrombus, the control comprising a control body having an inlet and an outlet connected by an aspiration passage configured to direct a fluid through the control, a sliding component configured to control a flow of fluid through the aspiration passage and configured for direct communication with a hand of a user, wherein the control may be placed in a first position by moving the sliding component in a first direction, and wherein the control may be placed in a second position by moving the sliding component in a second direction opposite the first direction.

Embodiment 26. The control of embodiment 25, wherein the inlet comprises a first luer fitting and the outlet includes a second luer fitting.

Embodiment 27. The control of embodiment 25, wherein the sliding component comprises a tactile surface for engaging the hand of a user.

Embodiment 28. The control of embodiment 27, wherein the tactile surface comprises one or more ridges protruding from the surface of the sliding component.

Embodiment 29. A control configured for use in a system for aspirating thrombus, the control comprising a control body having an inlet and an outlet connected by an aspiration passage, a rotary valve disposed at least partially within the aspiration passage and configured to control a flow of fluid through the aspiration passage, and a control interface configured for manipulation by a user to move the rotary valve between an open position and a closed position.

Embodiment 30. The control of embodiment 29, wherein the rotary valve comprises a ball valve or a stopcock.

Embodiment 31. The control of embodiment 29, wherein an angle of rotation of the rotary valve between the open position and the closed position is between approximately 45° and approximately 360°, or approximately 90° and approximately 225°, or approximately 135° and approximately 180°, or a range having any two of the foregoing as endpoints.

Embodiment 32. The control of embodiment 29, wherein the rotary valve comprises a quarter-turn ball valve.

Embodiment 33. The control of embodiment 32, wherein the control interface comprises a lever.

Embodiment 34. A control configured for use in a system for aspirating thrombus, the control having an open position and a closed position, the control comprising a control body having a channel disposed therein configured to receive tubing of an aspiration catheter, a first anvil disposed on or near a first side of the channel and a second anvil disposed on or near a second side of the channel opposite the first side of the channel, a control interface configured for manipulation by a user to move the control body between the open position and the closed position, and wherein a distance between the first and second anvils decreases to obstruct flow of a fluid through a lumen of tubing disposed within the channel when the control is moved to the closed position.

Embodiment 35. The control of embodiment 34, wherein the control interface comprises a button.

Embodiment 36. A system for aspirating thrombus, comprising an aspiration catheter comprising an elongate shaft configured for placement within a blood vessel of a subject, a supply lumen and an aspiration lumen each extending along the shaft, and an opening at or near a distal end of the supply lumen, the opening configured to allow injection of pressurized fluid into the aspiration lumen at or near the distal end of the aspiration lumen when the pressurized fluid is caused or allowed to flow through the supply lumen, a pressurized fluid source in fluid communication with the supply lumen to provide pressurized fluid to the supply lumen, a vacuum source in fluid communication with the aspiration lumen, and a control configured to regulate application of vacuum to the aspiration catheter, having an open and closed position, the control comprising a control body having an inlet and an outlet connected by an aspiration passage, a valve disposed at least partially within the aspiration passage, an occlusion member disposed at least partially within the control body, wherein the occlusion member is configured to enter the valve and obstruct flow of a fluid through the aspiration passage, a control interface configured to be manipulated by a user to move the control between the open and closed positions, and a return mechanism configured to facilitate movement of the occlusion member into the valve.

Embodiment 37. The system of embodiment 36, wherein the occlusion member applies a force to the occlusion member to maintain the valve in the closed position.

Embodiment 38. The system of embodiment 36 or 37, wherein the return mechanism is disposed between the occlusion member and the control body.

Embodiment 39. The system of any one of embodiments 36 through 38, wherein the return mechanism is adjacent a first end of the occlusion member opposite a second end of the occlusion member that enters the valve.

Embodiment 40. The system of any one of embodiments 36 through 39, wherein the return mechanism comprises a return spring.

Embodiment 41. The system of embodiment 40, wherein the return spring comprises a compression spring.

Embodiment 42. The system of embodiment 40, wherein the return spring comprises an extension spring.

Embodiment 43. The system of embodiment 40, wherein the return spring comprises a compression spring, an extension spring, a torsion spring, a wave disk spring, a volute spring, a spiral spring, or leaf spring.

Embodiment 44. The system of any one of embodiments 36 through 39, wherein the return mechanism comprises a solenoid.

Embodiment 45. The system of embodiment 44, further comprising an engagement pin disposed within the solenoid.

Embodiment 46. The system of embodiment 45, wherein the engagement pin is configured to push the occlusion member into the valve upon activation of the solenoid.

Embodiment 47. The system of embodiment 45, wherein the engagement pin is affixed to the occlusion member.

Embodiment 48. The system of embodiment 45, wherein the solenoid is affixed to the occlusion member.

Embodiment 49. The system of any one of embodiments 36 through 39, wherein the return mechanism comprises a pneumatic connection.

Embodiment 50. The system of embodiment 49, wherein the control body comprises a pressure chamber, wherein the pressure chamber is adjacent a first end of the occlusion member opposite a second end of the occlusion member that is configured to enter the valve.

Embodiment 51. The system of embodiment 49 or 50, wherein the control interface comprises a venting actuator.

Embodiment 52. The system of any one of embodiments 44 through 51, further comprising a spring configured to move the occlusion member out of the valve.

Embodiment 53. The system of any one of embodiments 36 through 52, wherein the control interface comprises a slider.

Embodiment 54. The system of any one of embodiments 36 through 52, wherein the control interface comprises a button.

Embodiment 55. The system of any one of embodiments 36 through 54, further comprising an electrical switch configured to be activated by the occlusion member when the occlusion member is moved to the open position.

Embodiment 56. The system of embodiment 55, wherein the electrical switch is configured to activate a vacuum source.

Embodiment 57. The system of embodiment 36, further comprising a handle having a longitudinal axis, wherein the control body comprises a longitudinal axis, and wherein the longitudinal axis of the handle is transverse to the longitudinal axis of the control body.

Embodiment 58. The system of embodiment 57, wherein an angle between the longitudinal axis of the handle and the longitudinal axis of the control body is within a range of approximately 45° to approximately 170°, or approximately 70° to approximately 150°, or approximately 90° to approximately 135°, or a range with any two of the foregoing as endpoints.

Embodiment 59. The system of embodiment 57, wherein the control body comprises a notch adjacent the handle configured to receive a purlicue of a hand of a user.

Embodiment 60. A system for aspirating thrombus, comprising an aspiration catheter comprising an elongate shaft configured for placement within a blood vessel of a subject, a supply lumen and an aspiration lumen each extending along the shaft, and an opening at or near a distal end of the supply lumen, the opening configured to allow injection of pressurized fluid into the aspiration lumen at or near the distal end of the aspiration lumen when the pressurized fluid is caused or allowed to flow through the supply lumen, a pressurized fluid source in fluid communication with the supply lumen to provide pressurized fluid to the supply lumen, a vacuum source in fluid communication with the aspiration lumen, and a control configured to regulate application of vacuum to the aspiration catheter, having an open and closed position, the control comprising a control body having a channel disposed therein configured to receive tubing of an aspiration catheter, a first anvil disposed on or near a first side of the channel and a second anvil disposed on or near a second side of the channel opposite the first side of the channel, a control interface configured for manipulation by a user to move the control body between the open position and the closed position, and wherein a distance between the first and second anvils decreases to obstruct flow of a fluid through the lumen of tubing disposed in the channel when the control is moved to the closed position.

Embodiment 61. The system of embodiment 60, wherein the control interface comprises a button.

Embodiment 62. A system for aspirating thrombus, comprising an aspiration catheter comprising an elongate shaft configured for placement within a blood vessel of a subject, a supply lumen and an aspiration lumen each extending along the shaft, and an opening at or near a distal end of the supply lumen, the opening configured to allow injection of pressurized fluid into the aspiration lumen at or near the distal end of the aspiration lumen when the pressurized fluid is caused or allowed to flow through the supply lumen, a pressurized fluid source in fluid communication with the supply lumen to provide pressurized fluid to the supply lumen, and a vacuum source in fluid communication with the aspiration lumen, a control configured to regulate application of vacuum to the aspiration catheter, having an open and closed position, the control comprising a control body having an inlet and an outlet connected by an aspiration passage, a valve disposed at least partially within the aspiration passage configured to be manually actuated by a user, an occlusion member disposed at least partially within the control body, wherein the occlusion member is configured to enter the valve and obstruct flow of a fluid through the aspiration passage, a control interface configured to be manipulated by a user to move the control between the open and closed positions.

Embodiment 63. The system of embodiment 62, wherein the valve further comprises a sliding component and wherein the control may be placed in a first position by moving the sliding component in a first direction and wherein the control may be placed in a second position by moving the sliding component in a second direction opposite the first direction.

Embodiment 64. The system of embodiment 63, wherein the inlet comprises a first luer fitting and the outlet includes a second luer fitting.

Embodiment 65. The system of embodiment 63, wherein the sliding component comprises a tactile surface for engaging a hand of a user.

Embodiment 66. The system of embodiment 65, wherein the tactile surface comprises one or more ridges protruding from the surface of the sliding component.

Embodiment 67. The system of embodiment 62, wherein the valve comprises a rotary valve.

Embodiment 68. The system of embodiment 67, wherein the rotary valve comprises a ball valve or a stopcock.

Embodiment 69. The system of embodiment 67, wherein an angle of rotation of the rotary valve between the open position and the closed position is between approximately 45° and approximately 360°, or approximately 90° and approximately 225°, or approximately 135° and approximately 180°, or a range having any two of the foregoing as endpoints.

Embodiment 70. The system of embodiment 67, wherein the rotary valve comprises a quarter-turn ball valve.

Embodiment 71. The system of embodiment 70, wherein the control interface comprises a lever.

What is claimed is:

1. A control configured for use in a system for aspirating thrombus, the control having an open position and a closed position, the control comprising:
a control body having an inlet and an outlet connected by an aspiration passage;
a valve disposed at least partially within the aspiration passage;
an occlusion member disposed at least partially within the control body, the occlusion member being configured to enter the valve and obstruct flow of a fluid through the aspiration passage;
a control interface configured to be manipulated by a user to move the control between the open and closed positions; and
a return mechanism configured to facilitate movement of the occlusion member into the valve.

2. The control of claim 1, wherein the return mechanism is:
disposed between the occlusion member and the control body; or
adjacent a first end of the occlusion member opposite a second end of the occlusion member that enters the valve.

3. The control of claim 1, wherein the return mechanism comprises a return spring.

4. The control of claim 3, wherein the return spring comprises a compression spring, an extension spring, a torsion spring, a wave disk spring, a volute spring, a spiral spring, or leaf spring.

5. The control of claim 1, wherein the return mechanism comprises a solenoid and an engagement pin disposed within the solenoid, wherein the engagement pin is configured to push the occlusion member into the valve upon activation of the solenoid.

6. The control of claim 5, wherein the solenoid or the engagement pin is affixed to the occlusion member.

7. The control of claim 1, wherein the return mechanism comprises a pneumatic connection.

8. The control of claim 7, wherein the control body comprises a pressure chamber, wherein the pressure chamber is adjacent a first end of the occlusion member opposite a second end of the occlusion member that is configured to enter the valve.

9. The control of claim 7, wherein the control interface comprises a venting actuator.

10. The control of claim 5, further comprising a spring configured to move the occlusion member out of the valve.

11. The control of claim 1, wherein the control interface comprises a slider or a button.

12. The control of claim 1, further comprising an electrical switch configured to be activated by the occlusion member when the occlusion member is moved to the open position.

13. The control of claim 12, wherein the electrical switch is configured to activate a vacuum source.

14. A control configured for use in a system for aspirating thrombus, the control having an open position and a closed position, the control comprising:
a control body having a longitudinal axis and configured to be moved between an open position and a closed position and having an aspiration passage;
a handle having a longitudinal axis transverse to the longitudinal axis of the control body;
a control interface configured to be manipulated by a user to move the control body between the open position and the closed position;
a valve disposed at least partially within the aspiration passage;
wherein the valve is configured to obstruct flow of a fluid through the aspiration passage when the control is in the closed position; and
wherein the valve is configured to allow flow through the aspiration passage when the control is in the open position.

15. The control of claim 14, wherein an angle between the longitudinal axis of the handle and the longitudinal axis of the control body is within a range of approximately 45° to approximately 170°, or approximately 70° to approximately 150°, or approximately 90° to approximately 135°, or a range with any two of the foregoing as endpoints.

16. The control of claim 14, wherein the control body comprises a notch adjacent the handle configured to receive a purlicue of a hand of a user.

17. A control configured for use in a system for aspirating thrombus, the control comprising:
a control body having an inlet and an outlet connected by an aspiration passage configured to direct a fluid through the control;
a sliding component configured to control a flow of fluid through the aspiration passage and configured for direct communication with a hand of a user;
wherein the control may be placed in a first position by moving the sliding component in a first direction; and
wherein the control may be placed in a second position by moving the sliding component in a second direction opposite the first direction.

18. The control of claim 17, wherein the inlet comprises a first luer fitting and the outlet includes a second luer fitting.

19. The control of claim 17, wherein the sliding component comprises a tactile surface for engaging the hand of a user.

20. The control of claim 19, wherein the tactile surface comprises one or more ridges protruding from the surface of the sliding component.
